SEQUENCE LISTING (18 sequences)

SEQ ID NO: 1 - protein, 155 aa, Artificial Sequence
  SYNTHETIC SEQUENCE
MAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNXYNTYRSRKYTSWYVALKRTGQYKLGSKTGPGQKAILFLPMSAKS

SEQ ID NO: 2 - protein, 155 aa, Artificial Sequence
  SYNTHETIC SEQUENCE
MAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNRYNTYRSRKYTSWYVALKRTGQYKLGSKTGPGQKAILFLPMSAKS

SEQ ID NO: 3 - protein, 155 aa, Artificial Sequence
  SYNTHETIC SEQUENCE
MAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNGYNTYRSRKYTSWYVALKRTGQYKLGSKTGPGQKAILFLPMSAKS

SEQ ID NO: 4 - protein, 155 aa, Artificial Sequence
  SYNTHETIC SEQUENCE
MAQGQITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNXYNTYRSRKYTSWYVALKRTGQYKLGSKTGPGQKAILFLPMSAKS

SEQ ID NO: 5 - protein, 155 aa, Artificial Sequence
  SYNTHETIC SEQUENCE
MAQGQITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNGYNTYRSRKYTSWYVALKRTGQYKLGSKTGPGQKAILFLPMSAKS

SEQ ID NO: 6 - protein, 145 aa, Artificial Sequence
  SYNTHETIC SEQUENCE
MQLYCRTGFHLEIFPNGTIQGTRKDHSRFGILEFISIAVGLVSIRGVDSGLYLGMNEKGELYGSEKLTQECVFREQFEENXYNTYSSNLYKHVDTGRRYYVALNKDGTPREGTRTKRHQKFTHFLPRPVDPDKVPELYKDILSQS

SEQ ID NO: 7 - protein, 127 aa, Artificial Sequence
  SYNTHETIC SEQUENCE
MQLYCRTGFHLEIFPNGTIQGTRKDHSRFGILEFISIAVGLVSIRGVDSGLYLGMNEKGELYGSEKLTQECVFREQFEENXYNTYSSNLYKHVDTGRRYYVALNKDGTPREGTRTKRHQKFTHFLPR

SEQ ID NO: 8 - protein, 145 aa, Artificial Sequence
  SYNTHETIC SEQUENCE
MQLYCRTGFHLEIFPNGTIQGTRKDHSRFGILEFISIAVGLVSIRGVDSGLYLGMNEKGELYGSEKLTQECVFREQFEEXWYNTYSSNLYKHVDTGRRYYVALNKDGTPREGTRTKRHQKFTHFLPRPVDPDKVPELYKDILSQS

SEQ ID NO: 9 - protein, 127 aa, Artificial Sequence
  SYNTHETIC SEQUENCE
MQLYCRTGFHLEIFPNGTIQGTRKDNSRFGILEFISIAVGLVSIRGVDSGLYLGMNEKGELYGSEKLTQECVFREQFEEXWYNTYSSNLYKHVDTGRRYYVALNKDGTPREGTRTKRHQKFTHFLPR

SEQ ID NO: 10 - DNA, 468 bp, Artificial Sequence
  SYNTHETIC SEQUENCE
atggctgccgggagcatcaccacgctgcccgcccttccggaggatggcggcagcggcgccttcccgcccgggcacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccggtacctggctatgaaggaagatggaagattactggcttctaaatgtgttacggatgagtgtttcttttttgaacgattggaatctaatnnntacaatacttaccggtctagaaaatacaccagttggtatgtggcattgaaacgaactgggcagtataaacttggttccaaaacaggacctgggcagaaagctatactttttcttccaatgtctgctaagagctga

SEQ ID NO: 11 - DNA, 468 bp, Artificial Sequence; identical to SEQ ID NO: 10
  SYNTHETIC SEQUENCE

SEQ ID NO: 12 - DNA, 468 bp, Artificial Sequence
  SYNTHETIC SEQUENCE
atggctgccgggagcatcaccacgctgcccgcccttccggaggatggcggcagcggcgccttcccgcccgggcacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccggtacctggctatgaaggaagatggaagattactggcttctaaatgtgttacggatgagtgtttcttttttgaacgattggaatctaatcgntacaatacttaccggtctagaaaatacaccagttggtatgtggcattgaaacgaactgggcagtataaacttggttccaaaacaggacctgggcagaaagctatactttttcttccaatgtctgctaagagctga

SEQ ID NO: 13 - DNA, 468 bp, Artificial Sequence
  SYNTHETIC SEQUENCE
atggctcangggcanatcaccacgctgcccgcccttccggaggatggcggcagcggcgccttcccgcccgggcacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccggtacctggctatgaaggaagatggaagattactggcttctaaatgtgttacggatgagtgtttcttttttgaacgattggaatctaatnnntacaatacttaccggtctagaaaatacaccagttggtatgtggcattgaaacgaactgggcagtataaacttggttccaaaacaggacctgggcagaaagctatactttttcttccaatgtctgctaagagctga

SEQ ID NO: 14 - DNA, 468 bp, Artificial Sequence
  SYNTHETIC SEQUENCE
atggctcangggcanatcaccacgctgcccgcccttccggaggatggcggcagcggcgccttcccgcccgggcacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccggtacctggctatgaaggaagatggaagattactggcttctaaatgtgttacggatgagtgtttcttttttgaacgattggaatctaatggntacaatacttaccggtctagaaaatacaccagttggtatgtggcattgaaacgaactgggcagtataaacttggttccaaaacaggacctgggcagaaagctatactttttcttccaatgtctgctaagagctga

SEQ ID NO: 15 - DNA, 438 bp, Artificial Sequence
  SYNTHETIC SEQUENCE
atgcagctatactgcaggactggatttcacttagaaatcttccccaatggtactatccagggaaccaggaaagaccacagccgatttggcattctggaatttatcagtatagcagtgggcctggtcagcattcgaggcgtggacagtggactctacctcgggatgaatgagaagggggagctgtatggatcagaaaaactaacccaagagtgtgtattcagagaacagttcgaagaaaacnnntataatacgtactcgtcaaacctatataagcacgtggacactggaaggcgatactatgttgcattaaataaagatgggaccccgagagaagggactaggactaaacggcaccagaaattcacacattttttacctagaccagtggaccccgacaaagtacctgaactgtataaggatattctaagccaaagttga

SEQ ID NO: 16 - DNA, 384 bp, Artificial Sequence
  SYNTHETIC SEQUENCE
atgcagctatactgcaggactggatttcacttagaaatcttccccaatggtactatccagggaaccaggaaagaccacagccgatttggcattctggaatttatcagtatagcagtgggcctggtcagcattcgaggcgtggacagtggactctacctcgggatgaatgagaagggggagctgtatggatcagaaaaactaacccaagagtgtgtattcagagaacagttcgaagaaaacnnntataatacgtactcgtcaaacctatataagcacgtggacactggaaggcgatactatgttgcattaaataaagatgggaccccgagagaagggactaggactaaacggcaccagaaattcacacattttttacctagatga

SEQ ID NO: 17 - DNA, 438 bp, Artificial Sequence
  SYNTHETIC SEQUENCE
atgcagctatactgcaggactggatttcacttagaaatcttccccaatggtactatccagggaaccaggaaagaccacagccgatttggcattctggaatttatcagtatagcagtgggcctggtcagcattcgaggcgtggacagtggactctacctcgggatgaatgagaagggggagctgtatggatcagaaaaactaacccaagagtgtgtattcagagaacagttcgaagaannntggtataatacgtactcgtcaaacctatataagcacgtggacactggaaggcgatactatgttgcattaaataaagatgggaccccgagagaagggactaggactaaacggcaccagaaattcacacattttttacctagaccagtggaccccgacaaagtacctgaactgtataaggatattctaagccaaagttga

SEQ ID NO: 18 - DNA, 384 bp, Artificial Sequence
  SYNTHETIC SEQUENCE
atgcagctatactgcaggactggatttcacttagaaatcttccccaatggtactatccagggaaccaggaaagaccacagccgatttggcattctggaatttatcagtatagcagtgggcctggtcagcattcgaggcgtggacagtggactctacctcgggatgaatgagaagggggagctgtatggatcagaaaaactaacccaagagtgtgtattcagagaacagttcgaagaannntggtataatacgtactcgtcaaacctatataagcacgtggacactggaaggcgatactatgttgcattaaataaagatgggaccccgagagaagggactaggactaaacggcaccagaaattcacacattttttacctagatga

(19) United States
(12) Patent Application Publication
(10) Pub. No.: US 2010/0274362 A1
(43) Pub. Date: Oct. 28, 2010

(54) Cartilage particle tissue mixtures optionally combined with a cancellous construct

(76) Inventors: Avner Yayon, Moshav Sitria (IL); Katherine G. Truncale, Hillsborough, NJ (US); Hilla Barkay-Olami, Ness Ziona (IL); Alex B. Callahan, Perth Amboy, NJ (US); Arthur A. Gertzman, Flemington, NJ (US); Yen-Chen Huang, East Brunswick, NJ (US); Morris L. Jacobs, Newtown, PA (US); John C. Munson, Easton, PA (US); Eric J. Semler, Piscataway, NJ (US); Roman Shikhanovich, Edison, NJ (US); Baruch Stern, Haifa (IL); Moon Hae Sunwoo, Old Tappan, NJ (US); William W. Tomford, Belmont, MA (US); Judith I. Yannariello-Brown, Somerset, NJ (US)

Correspondence Address: GREENBERG TRAURIG, LLP, 200 PARK AVE., P.O. BOX 677, FLORHAM PARK, NJ, 07932, US

(21) Appl. No.: 12/657,207

(22) Filed: Jan. 14, 2010

Related U.S. Application Data
(60) Provisional application No. 61/205,436, filed on Jan. 15, 2009.

(51) Int. Cl. A61F 2/02; A61K 9/19; A61K 9/00; A61K 38/18; A61P 19/00

(52) U.S. Cl. 623/23.72; 424/499; 424/484; 514/9.1; 514/8.9; 514/8.8; 514/8.2; 514/8.1; 514/8.6; 514/9.2

(57) ABSTRACT

Mixtures, such as gels or pastes, comprising freeze-milled cartilage particles and exogenous growth factors are used for repairing chondral defects. Such mixtures may be applied to constructs comprising cancellous bone for implantation at the defect site. Suitable growth factors include variants of FGF-2, particularly variants that include a sole amino acid substitution for asparagine at amino acid 111 of the β8-β9 loop of the FGF-2 peptide. Such FGF-2 variants are released slowly and continuously at a constant rate from cartilage pastes. In other embodiments, the amino acid substituted for asparigine is glycine. Other variants that may be used include FGF-9 variants having truncated chains and a sole amino acid substitution in the β8-β9 loop of the FGF-9 peptide either for tryptophan at amino acid 144 or for asparagine at amino acid 143.

### CROSS-REFERENCE TO RELATED APPLICATIONS

The present application claims benefit of U.S. Provisional Patent Application No. 61/205,436, filed on Jan. 15, 2009, which is incorporated by reference herein in its entirety.

### STATEMENT REGARDING FEDERALLY-SPONSORED RESEARCH OR DEVELOPMENT

None.

### REFERENCE TO SEQUENCE LISTING, TABLE OR COMPUTER PROGRAM LISTING COMPACT DISC APPENDIX

None.

### FIELD OF THE INVENTION

The present invention relates to preparations and constructs for use in repairing defects in cartilage and/or bone, and, more particularly, to combinations of cartilage particle mixtures with exogenous growth factors and combinations of such cartilage particle mixtures and exogenous growth factors with constructs comprising bone.

### BACKGROUND OF THE INVENTION

Chondrogenesis is the process of growth and differentiation of cartilage cells (chondrocytes), leading to the proliferation of such cells and the development of a robust, specialized extracellular matrix surrounding such cells. Cartilage is the specialized matrix of chondrocytes and particular cartilage extracellular matrix components surrounding such chondrocytes. Disordered growth and repair of cartilage cells results in tissue with primarily fibrotic morphology, as opposed to the cartilage extracellular matrix resulting from normal growth and development of chondrocytes and having characteristic proteoglycan and collagen II components.

Articular cartilage injury and degeneration present medical problems to the general population which are constantly being addressed by orthopedic surgeons. Thousands of arthroplastic and joint repair procedures are performed every year in the United States, including total hip and total knee arthroplasties and open arthroscopic procedures to repair cartilaginous defects of the knee.

Reference is now made to FIG. 1, which illustrates a knee joint having articular cartilage tissue forming a lining which faces the joint cavity on one side, and is linked to the subchondral bone plate by a narrow layer of calcified cartilage tissue on the other side. Articular cartilage consists primarily of extracellular matrix with a sparse population of chondrocytes distributed throughout the tissue. Articular cartilage comprises chondrocytes, type II collagen fibril meshwork, proteoglycans, and water. Active chondrocytes are unique in that they have a relatively low turnover rate and are sparsely distributed within the surrounding matrix. The collagens give the tissue its form and tensile strength and the interaction of proteoglycans with water gives the tissue its stiffness to compression, resilience and durability. The articular cartilage provides a low friction bearing surface over the bony parts of the joint. If the lining becomes worn or damaged resulting in lesions, joint movement may be painful or severely restricted. Whereas damaged bone typically can regenerate successfully, articular cartilage regeneration is quite limited because of its limited regenerative and reparative abilities.

Articular cartilage lesions generally do not heal, or heal only partially under certain biological conditions, due to the lack of nerves, blood vessels and a lymphatic system. The limited reparative capabilities of articular cartilage usually results in the generation of repair tissue that lacks the structure and biomechanical properties of normal articular cartilage. Generally, the healing of the defect results in a fibrocartilaginous repair tissue that lacks the structure and biomedical properties of articular cartilage and degrades over the course of time. Articular cartilage lesions are frequently associated with disability and with symptoms such as joint pain, locking phenomena and reduced or disturbed function. These lesions are difficult to treat because of the distinctive structure and function of articular cartilage. Such lesions are believed to progress to severe forms of osteoarthritis. Osteoarthritis is the leading cause of disability and impairment in middle-aged and older individuals, entailing significant economic, social and psychological costs. Each year, osteoarthritis accounts for millions of physician visits and thousands of hospital admissions.

### BRIEF SUMMARY OF THE INVENTION

In one aspect, the present invention is directed towards a cartilage particle mixture for repairing a chondral defect, comprising freeze-milled cartilage particles and exogenous FGF-2 of FGF-9 variants. In some embodiments, an FGF-2 variant includes a sole amino acid (asparagine) substitution at amino acid 111 of the β8-β9 loop of the peptide, as shown and described in U.S. Pat. No. 7,563,769, issued on Jul. 21, 2009 or International (PCT) Publication No. WO 2008/038287, published Apr. 3, 2008. In some embodiments, the amino acid substituted for asparagine is arginine. In other embodiments, the amino acid substituted for asparigine is glycine. In other embodiments, an FGF-9 variant includes a sole amino acid (tryptophan) substitution at amino acid 144 (“W144”) of the β8-β9 loop of the peptide, or a sole amino acid (asparagine) substitution at amino acid 143 (“N143”) of the β8-β9 loop of the peptide, as also shown and described in U.S. Pat. No. 7,563,769, issued on Jul. 21, 2009. In some of such embodiments, the amino acid substituted for tryptophan at W144 is selected from among glycine (Gly, G), arginine (Arg, R), glutamate (Glu, E) and valine (Val, V), and the amino acid substituted for asparagine at N143 is serine. In some embodiments, the cartilage particle mixture is combined with a construct including demineralized cancellous bone. In other aspects of the invention, such cartilage particle mixtures are used in a method comprising the step of administering the cartilage particle mixture to a patient in need of treatment, wherein the administered cartilage particle mixture subsequently generates repair tissue that contains components comprising glycosaminoglycans and collagen II. In some embodiments of the method, the cartilage particle mixture is applied to the defect. In other embodiments of the method, the cartilage particle mixture is applied to a construct including demineralized and non-demineralized cancellous bone and/or cortical bone, which is inserted into the defect.

### BRIEF DESCRIPTION OF THE DRAWINGS

This patent or patent application publication contains at least one drawing executed in color, which includes a color photograph. Copies of this patent or patent application publication with color drawings will be provided by the Office upon request and payment of the necessary fee.

The present invention will be further explained with reference to the attached drawings. The drawings shown are not necessarily to scale, with emphasis instead generally being placed upon illustrating the principles of the present invention.

FIG. 1 is an anatomical illustration of a knee joint having articular cartilage in which a lesion has formed;

FIG. 2 is a chart depicting an alignment of FGF genes;

FIGS. 3A and 3B are photographic depictions of the porosity of a component of a cancellous construct produced in accordance with an embodiment of the present invention;

FIG. 4 is a depiction of nanograms of endogenous TGF-β1 per gram of cartilage particles isolated from said cartilage particles of several subjects through guanidine HCl extraction and subsequent dialysis;

FIG. 5 is a comparison of relative amounts (nanograms) of endogenous TGF-β1 per gram of cartilage particles isolated from minced and freeze-milled cartilage through guanidine HCL extraction and subsequent dialysis;

FIG. 6 is a depiction of picograms of endogenous FGF-2 per gram of cartilage particles isolated from freeze-milled cartilage particles of several tissue donors through guanidine HCl extraction and subsequent dialysis;

FIG. 7 is a depiction of nanograms of endogenous BMP-2 per gram of cartilage particles isolated from freeze-milled cartilage particles of several tissue donors through guanidine HCl extraction and subsequent dialysis;

FIG. 8 is a depiction of nanograms of endogenous BMP-14 (GDF-5) per gram of cartilage particles isolated from freeze-milled cartilage particles of several tissue donors through guanidine HCl extraction and subsequent dialysis;

FIG. 9 is a depiction of nanograms of endogenous IGF-1 per gram of cartilage particles isolated from freeze-milled cartilage particles of several tissue donors through guanidine HCl extraction and subsequent dialysis;

FIG. 10 is a view of newly synthesized articular cartilage from in vivo experimentation, demonstrating infiltration of lacunae by chondrocytes;

FIGS. 11A and 11B are views of collagen immunohistochemistry staining for collagen II, a marker of articular cartilage, showing that both cartilage particles and newly-synthesized extracellular matrix stain positive for collagen II;

FIG. 12 is a pictorial depiction of a cancellous construct, for example, of the type disclosed in the instant application;

FIG. 13 demonstrates homogenous distribution of the cartilage particles in a cap portion of the construct, as indicated by positive proteoglycan (Safranin-O) staining;

FIGS. 14 A-14 H demonstrate relative chondrogenesis over a period of 24 weeks post-treatment: FIGS. 14 A, 14 C, 14 E, and 14 G are Safranin-O stained for proteoglycan assessment; FIGS. 14 B, 14 D, 14 F, and 14 H are anti-collagen II stained for collagen II assessment; FIGS. 14 A and 14 B represent microfracture; FIGS. 14 C and 14 D represent empty defect; FIGS. 14 E and 14 F represent a construct without cartilage particles; and FIGS. 14 G and 14 H represent a construct in combination with (i.e., loaded with) freeze-milled cartilage particles;

FIG. 15 is a chart illustrating the release of an FGF variant over time from freeze-milled cartilage particles; and

FIG. 16 is a chart comparing the total amount of the FGF variant of FIG. 15 released from freeze-milled cartilage particles with the amount of released FGF variant that promotes cell proliferation.

FIG. 17 is a chart illustrating the release of an FGF variant over time from freeze-milled cartilage particles at two temperatures;

FIG. 18 is a chart illustrating the release of an FGF variant over time from freeze-dried cartilage paste at two temperatures;

FIG. 19 is a chart comparing the total amount of the FGF variant of FIG. 18 released from freeze-milled cartilage particles with the amount of released FGF variant that promotes cell proliferation;

FIG. 20 is a chart illustrating the release of an FGF variant over time from reconstituted freeze-dried cartilage paste at two temperatures;

FIG. 21 is a chart comparing the total amount of the FGF variant of FIG. 20 released from freeze-milled cartilage particles with the amount of released FGF variant that promotes cell proliferation;

FIG. 22 is a chart illustrating the release of an FGF variant over time from a mixture including the FGF variant, hyaluronic acid and cartilage particles after freeze-drying;

FIG. 23 is a chart illustrating the release of an FGF variant over time from different preparations of cartilage paste;

FIG. 24 is another chart illustrating the release of an FGF variant over time from other different preparations of cartilage paste;

FIG. 25 is a chart illustrating the release of an FGF variant over time from different preparations of cartilage paste using goat cartilage particles;

FIG. 26 is a chart illustrating the release of an FGF variant over time from different preparations of reconstituted freeze-dried cartilage paste;

FIG. 27 is a chart illustrating the stability of an FGF variant over time in a solution containing lyophilized cartilage particles;

FIG. 28 is a chart illustrating the release of an FGF variant over time from a group of solutions containing lyophilized cartilage particles and hyaluronic acid, and having different temperatures and storage times;

FIG. 29 is another chart illustrating the release of an FGF variant over time from a group of solutions containing lyophilized cartilage particles and hyaluronic acid, and having different temperatures and storage times;

FIG. 30 is a chart illustrating the release of an FGF variant over time from a group of solutions containing lyophilized human cartilage particles combined with a fibrinogen scaffold;

FIG. 31 is a chart illustrating the release of an FGF variant over time from a group of solutions containing goat cartilage particles combined with a fibrinogen scaffold;

FIG. 32 is another chart illustrating the release of an FGF variant over time from a group of solutions containing goat cartilage particles combined with a fibrinogen scaffold;

FIG. 33 is a chart illustrating the release of an FGF variant over time from a group of cancellous bone pieces; and

FIG. 34 is a chart illustrating the growth of cells seeded on cancellous bone pieces.

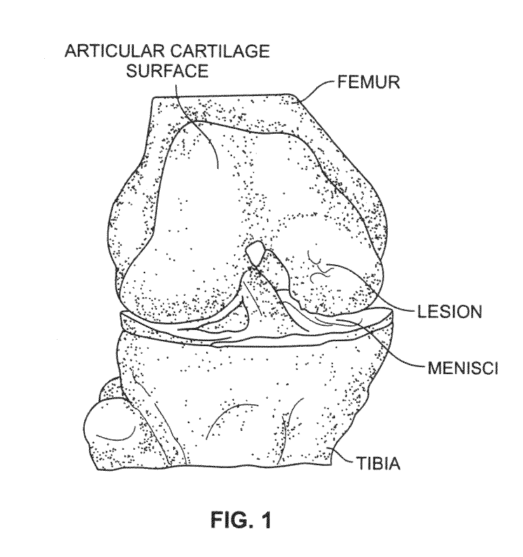

While the above-identified drawings set forth presently disclosed embodiments, other embodiments are also contemplated, as noted in the detailed description. This disclosure presents illustrative embodiments by way of representation and not limitation. Numerous other modifications and embodiments can be devised by those skilled in the art which fall within the scope and spirit of the principles of the presently disclosed invention.

### DETAILED DESCRIPTION OF THE INVENTION

The scope of the present invention encompasses combinations of cartilage particle mixtures with exogenous growth factors and combinations of such cartilage particle mixtures and exogenous growth factors with constructs comprising demineralized cancellous bone, methods of making such cartilage particle mixtures and constructs, and the uses of such cartilage particle mixtures, exogenous growth factors, and constructs in repairing osteochondral defects. Detailed embodiments of the present invention are disclosed herein, however, it is to be understood that all such disclosed embodiments are merely illustrative of the invention, which may be embodied in various forms. In addition, each of the examples given in connection with the various embodiments of the invention is intended to be illustrative, and not restrictive. Further, the figures are not necessarily to scale, and some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

The term “active” or “activated” and its various grammatical forms, as used herein, refers to having biological or physiological effect.

The term “antagonist”, as used herein, refers to a substance that counteracts the effects of another substance. The term “agonist” as used herein refers to a chemical substance capable of activating a receptor to induce a full or partial pharmacological response.

The terms “autologous” and “autograft”, as used herein, refer to transplanted or implanted tissue or cells which originate with or are derived from the recipient thereof. The terms “allogeneic” or “allograft” refer to transplanted or implanted tissue or cells which originate with or are derived from a donor of the same species as the recipient thereof. The terms “xenogeneic” or “xenograft” refer to transplanted or implanted tissue or cells which originate with or are derived from a species other than the recipient thereof.

The term “biocompatible”, as used herein, refers to materials which have low toxicity, acceptable foreign body reactions within the living body, and/or affinity with living tissues.

The term “cartilage”, as used herein, refers to a specialized type of connective tissue that contains chondrocytes embedded in an extracellular matrix. The biochemical composition of cartilage differs according to type, but in general comprises collagen, predominately type II cartilage along with other minor types (e.g., types IX and XI), proteoglycans, other proteins, and water. Several types of cartilage are recognized in the art, including, e.g., hyaline cartilage, articular cartilage, costal cartilage, fibrous cartilage, meniscal cartilage, elastic cartilage, auricular cartilage, and yellow cartilage. The production of any type of cartilage is intended to fall within the scope of the invention.

The term “chondrocytes”, as used herein, refers to cells which are capable of producing components of cartilage tissue.

The term “conserved segment”, as used herein refers to similar or identical sequences that may occur within nucleic acids, proteins or polymeric carbohydrates within multiple species of organism or within different molecules produced by the same organism.

The term “construct”, as used herein, refers to a device that includes one or more structural components which are constructed from milled pieces of bone, or other biocompatible materials, and is made to be implanted at the site of a tissue defect (e.g., an articular cartilage defect).

The term “demineralized bone”, as used herein, refers to bone whose native mineral content has been removed, e.g., by soaking the bone in a dilute mineral acid such as hydrochloric acid (HCl). In some embodiments of the present invention, demineralized bone has a calcium content of less than 0.5% by weight.

The term “homology”, as used herein, refers to the quality of being similar or corresponding in position, value, structure or function.

The term “isoform”, as used herein, refers to any of a group of two or more different proteins that are produced by different genes but have similar function and similar sequence. For example, fibroblast growth factor (FGF) has several isoforms including, but not limited to, FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, and FGF-23, and recombinants and variants thereof.

The terms “freeze-milling” or “freezer-milling”, as used herein, refer to a process wherein a tissue (e.g., cartilage) is cryogenically frozen (e.g., by use of a liquefied gas freezing agent such as liquid nitrogen or liquid helium) and then ground into particles.

The terms “lyophilize” or “freeze-drying”, as used herein, refer to the preparation of a composition in dry form by rapid freezing and dehydration in the frozen state (sometimes referred to as sublimation). The process may take place under vacuum at reduced air pressure, resulting in drying at a lower temperature than required at full air pressure.

The term “native”, as used herein, refers to the typical form of an organism, strain, gene, protein, nucleic acid, or characteristic as it occurs in nature. Wild-type refers to the most common phenotype in the natural population. The terms “native”, “wild-type” and “naturally occurring” are used interchangeably.

The term “plasma”, as used herein, refers to the fluid, non-cellular portion of the blood of humans or other animals as found prior to coagulation.

The term “plasma protein”, as used herein, refers to the soluble proteins found in the plasma of normal specimens of humans or other animals. Such proteins include, but are not limited to coagulation proteins, albumin, lipoproteins and complement proteins.

The term “recombinant”, as used herein, refers to a cell or vector that has been modified by the introduction of a heterologous nucleic acid or the cell that is derived from a cell so modified. “Recombinant growth factors” are growth factors produced by such recombinant cells as a result of such modifications that are not produced in the same amounts, or not produced at all, by the native form of the cell, or growth factors homologous to the aforesaid growth factors, but produced by other means. The term “recombinant” as used herein does not encompass the alteration of the cell or vector by naturally occurring events (e.g., spontaneous mutation, natural transformation/transduction/transposition) such as those occurring without deliberate human intervention.

The term “reference sequence”, as used herein, refers to a sequence used as a basis for sequence comparison. A reference sequence may be a subset or the entirety of a specified sequence; for example, as a segment of a full-length cDNA or gene sequence, or the complete cDNA or gene sequence.

The term “substitution”, as used herein, refers to that in which an amino acid or amino acids are exchanged for another amino acid or amino acids in the polypeptide or protein. With respect to DNA, “substitution” is also used herein to refer to that in which a base or bases are exchanged for another base or bases in the DNA. The term “similar” is used interchangeably with the terms analogous, comparable, or resembling, meaning having traits or characteristics in common.

The term “tissue” is used in the general sense herein to mean any transplantable or implanted biological tissue, the survivability of which is improved by the methods described herein upon implantation. In particular, the overall durability and longevity of the implant are improved, and host-immune system mediated responses are substantially eliminated.

The terms “transplant” and “implant”, as used herein, refer to tissue, material or cells (autograft, allograft, or xenograft) which may be introduced into the body of a patient.

The terms “variants” and “mutants” are used herein with respect to polypeptides and proteins to refer to amino acid sequences with substantial identity to a reference amino acid sequence. The differences in the sequences may be the result of changes, either naturally or by design, in sequence or structure. The terms “variants”, “mutants”, and “derivatives” are used herein with respect to DNA to refer to nucleotide sequences with substantial identity to a reference nucleotide sequence. Again, the differences in the sequences may by the result of changes, either naturally or by design, in sequence or structure.

The single letter designation for amino acids is used predominately herein. As is well known by one of skill in the art, such single letter designations are as follows: A is alanine; C is cysteine; D is aspartic acid; E is glutamic acid; F is phenylalanine; G is glycine; H is histidine; I is isoleucine; K is lysine; L is leucine; M is methionine; N is asparagine; P is proline; Q is glutamine; R is arginine; S is serine; T is threonine; V is valine; W is tryptophan; and Y is tyrosine.

The single letter designation for purines and pyrimidines is used herein. Purines include adenine (A), and guanine (G); pyrimidines include cytosine (C), thymine (T), and uracil (U).

### Cancellous Constructs

Cartilage repair constructs (e.g., scaffolds or implants) or their components may be made of allograft cancellous bone. Constructs or their components may also be made of allograft cortical bone and/or xenograft bone when the same is properly treated, or from other biocompatible materials. Cancellous bone is preferred because its porous structure enables it to act as a natural matrix for receiving and retaining therein a mixture containing cartilage particles and various bioactive chondrogenic materials for the repair of articular cartilage defects. Cancellous bone also acts as a conduit for tissue ingrowth and regeneration.

One example of a multi-piece cancellous bone construct for implantation into the site of a cartilage defect includes a base member and a cap member that is held fixed in place in relation to the base member. Such a cancellous bone construct, which is disclosed in U.S. Patent Application Publication No. 2008/0255676, published on Oct. 16, 2008, the disclosure of which is incorporated herein by reference in its entirety, includes a cap member at least partially constructed of demineralized cancellous bone, and a base member at least partially constructed of non-demineralized (i.e., mineralized) cancellous bone. The disclosures made herein are made in relation to the construct of the aforementioned exemplary construct, but persons skilled in the art will recognize that the disclosed materials and methods may be adapted to other constructs, scaffolds and implants comprising bone or other biocompatible materials.

The cancellous bone composition of the base member of the construct is similar to that of the surrounding subchondral bone into which it is to be implanted. The base member provides mechanical support to the cap member, thereby enabling the construct to act as a load-bearing scaffold. In addition, the cancellous bone of the base member is porous, thereby enabling blood from the adjacent subchondral bone to permeate rapidly throughout the construct, providing the host cells necessary for healing.

In one aspect of the invention, the cancellous base member of the construct presents a structural, osteoinductive matrix through which new bone is formed. The high degree of porosity of the cancellous bone allows for rapid penetration of blood, nutrients, and cells from the surrounding bleeding bone environment. This was observed during implantation of the construct in a critical sized in vivo goat osteochondral defect. In another aspect of the invention, the demineralized cap has been rendered non-osteoinductive through chemical treatment (e.g., by soaking in hydrogen peroxide solution), by thermal treatment, or by irradiation.

The porous, three-dimensional nature of the cancellous bone also provides considerable surface area for cellular attachment throughout the construct, including in the base member. Bone healing occurs through a process of bone resorption followed by new bone formation. Here, the presence of the acellular, non-demineralized bone of the base member triggers a biologic response in which osteoclasts begin to break down the implanted bone matrix. This event then leads to the activation of osteoblasts, via paracrine signaling, which starts to deposit new bone matrix. The final result of this ongoing remodeling is a de novo cancellous bone structure that is fully integrated into the subchondral bone at the defect site.

As discussed above, the high degree of porosity of cancellous bone also allows cartilage particle mixtures, such as the gels and pastes described herein, to be loaded into and/or applied to constructs made of such bone. In an embodiment of the present invention, a cartilage paste may be loaded into a construct according to the following protocol:

(1) freeze-milled allograft cartilage particles that were processed from the same donor are weighed and transferred to a small mixing jar;

(2) 0.78 cc of phosphate buffer saline (PBS) solution are added for each 0.22 g of cartilage particles, and the solution is stirred with a spatula to create a paste-like mixture;

(3) the mixture is transferred to a 10 ml syringe and allowed to equilibrate for five to ten minutes, with a syringe cap preferably used to cover the tip of the syringe and prevent the mixture from drying out;

(4) the assembled construct is placed in a paste-loading fixture and a small portion of the cartilage paste mixture is dispensed onto the top of the assembled construct (e.g., the top section of the cap member);

(5) a large spatula is used to spread the cartilage paste mixture throughout the cap member; and

(6) excess paste is wiped off and a smooth surface is created on the top surface and the outer curved wall of the cap member.

The quantity of cartilage particle mixture deposited onto the construct depends on a variety of factors that may be appreciated by those skilled in the art, including, for example, the dimensions of the construct, the viscosity and density of the cartilage particle mixture, the size of the cartilage particles, the anatomical and/or physical properties of the allograft tissues from which the construct and cartilage particles are derived, etc.

In an embodiment of the present invention, the surgical repair of a cartilage or osteochondral defect using the construct may be performed according to the following operation. A surgeon debrides (e.g., shaves away) the damaged or diseased portion of cartilage and the underlying subchondral bone from an articular cartilage defect area. A defect area bore is cut in the patient's articular cartilage layer and underlying subchondral bone layer. The defect removal and bore creation may be performed using a flat-bottom drill. The subchondral bone that is exposed by the creation of the bore may then be subjected to a microfracture procedure, whereby the surgeon uses an awl to create a number of small portals in the subchondral bone, causing it to bleed into the bore.

The surgeon may then modify the size and/or shape of the construct for implantation into the bone. For example, the surgeon may chamfer the bottom end of the base member to facilitate insertion of the construct into the bore. The bottom end of the base member may also be trimmed by the surgeon to shorten the height of the construct, thereby matching the construct to the bore, if the bore depth is less than the original height of the construct.

The construct is then implanted into the bore in a dry (i.e., lyophilized) state. Once inserted, the construct is re-hydrated by the bleeding from the surrounding host tissue (e.g., the cartilage and the subchondral bone). The construct may also be re-hydrated by the bleeding bone portals if the surgeon performed the aforementioned microfracture procedure. The construct may also be rehydrated in a solution such as saline prior to implantation.

The construct may be placed in the bore so that the top surface of the cap member is substantially flush with the surface of the patient's adjacent articular cartilage to form a smooth, continuous load-bearing surface. The bottom end of the base member may be supported by a bottom surface of the bore. The construct may have a diameter substantially equal to the diameter of the bore, in order to create an interference fit there between (e.g., an interference fit with the surrounding walls of the bore). Alternatively, the construct may have a diameter that is larger than the diameter of the bore, in order to create a press-fit therewith. Affixation and/or suitable glue materials may be used, for example, to seal the cartilage particles in the construct and to prevent synovial fluid infiltration, and/or, for example, to affix the construct in place within the bore post-implantation.

It is envisioned that cells (e.g., bone marrow cells, stem cells, progenitor cells and chondrocytes) may be inserted into the cap member and/or the entire construct before the construct is implanted into the defect area. The desired cellular density of the cells in the construct is a therapeutically effective density.

The construct may also be modified to include regionally-specific chondrogenic and osteogenic regions in the cap member and the base member, respectively. More particularly, the cap member may incorporate cartilage particles (e.g., in a mixture) and/or chondrogenic growth factors as described herein, and the base member may incorporate demineralized bone matrix and/or osteogenic growth factors. Alternatively, as growth factor activity is often context-dependent, a single growth factor having environmentally-specific activity may be incorporated in both the base member and the cap member. In another embodiment, any combination of chondrogenic and/or osteogenic growth factors may be employed. Growth factors may be incorporated into the cartilage particle mixture before the mixture is applied to the construct, or may be applied to the construct before the cartilage particle mixture is applied.

### Cartilage Particles

In an embodiment, the cartilage particles described herein are administrable as a stand-alone therapeutic treatment.

In an embodiment, the cartilage particles described herein are milled allograft cartilage particles. In an embodiment, allograft cartilage particles are milled, e.g. by use of a freeze-milling (i.e., freezer-milling) process wherein the cartilage is cryogenically frozen, for example by use of a liquefied gas freezing agent (e.g., liquid nitrogen or liquid helium) and then ground into particles.

In an embodiment, a cartilage defect repair material includes the aforementioned freeze-milled cartilage particles. In an embodiment, the cartilage defect repair material and the freeze-milled cartilage particles are sterile.

In another embodiment, the cap member is infused with a mixture such as a paste or gel that includes freeze-milled allograft cartilage particles. The term “gel” refers to a mixture of freeze-milled cartilage in a biocompatible carrier having a viscosity which is less than and is less rigid than a mixture of freeze-milled cartilage referred to by the terms “putty” or “paste” and contains less cartilage by weight than putty or paste. The cartilage paste or gel components are believed to provide the environmental and biochemical cues to elicit a healing response from the cells. For example, paste or gel components such as proteoglycans, collagen type II and other extracellular matrix components and their substituents may be present in greater concentration and/or bioavailability as a function of the processing of freeze-milled cartilage (e.g., freeze-milling cartilage may result in cartilage particles that are characterized as having greater exposure/bioavailability of different cytokines, growth factors, etc. to the surrounding environment). These available factors may then exert effects on cells that have infiltrated the construct from the surrounding host tissue and bleeding bone, synovium, etc. In an embodiment, the cells are chondrocytes. In another embodiment, the cells are capable of differentiation into chondrocyte lineage. In another embodiment, the cells are mesenchymal stem cells. Further examples include, without limitation, pluripotent stem cells; progenitor cells; mesenchymal stem and progenitor cells; stromal cells; and cartilage stem cells.

The cartilage particles may be irregularly shaped, and are passed through a sieve having 212 micron openings. While at least one dimension of each of the particles will be 212 microns or smaller in order to fit through the sieve, certain other axis lengths of the same particles may be greater than 212 microns, rendering the particles unable to pass through the sieve openings in that particular orientation. Several differently-sized cartilage particles are described in U.S. Pat. No. 7,067,123; issued on Jun. 27, 2006, which is incorporated by reference herein in its entirety.

In an embodiment, the cartilage particles have a size within a range of from about 10 microns to about 210 microns (i.e., from about 0.01 mm to about 0.21 mm). Alternatively, the cartilage particles may have a size (i.e., the aforesaid at least one dimension) that is within a range of from about 10 microns to about 120 microns (i.e., from about 0.01 mm to about 0.12 mm). The aforesaid at least one dimension of the cartilage particles may alternatively be less than or equal to 212 microns; within a range of from about 5 microns to about 212 microns; within a range of about 6 microns to about 10 microns; less than or equal to 5 microns; less than or equal to 10 microns; or less than or equal to 100 microns. In an embodiment, the aforesaid at least one dimension of most of the cartilage particles is less than 100 microns. In another embodiment, the aforesaid at least one dimension of the cartilage particles has a mean and/or median value in the range of between 10 microns and 200 microns. The small size of the cartilage particles can facilitate the increased exposure of or release of various growth factors due to the increased aggregate surface area of the particulate cartilage used, and can increase the capacity of the surrounding and infiltrating cells to attach to the cartilage particles.

In another embodiment, the cartilage particle size may facilitate the stable infiltration of the porous, demineralized portion of the construct by the cartilage particles. In an embodiment, the cartilage particles are freeze-milled to a size that permits them to be inserted into and retained by the pores in the cancellous bone of the cap member while optimizing the packing density of the particles therein.

The porosity of the cap member and cartilage particle size and/or shape may synergistically facilitate retention of the cartilage particles within the construct. Other factors facilitating retention of the cartilage particles in the construct throughout a range of motion include, but are not limited to, construct porosity, cartilage particle size and/or shape, construct and/or cartilage particle co-administered agents, moisture content of the construct and/or cartilage particles, blood clotting processes in an area of bleeding bone or other tissue proximate to the inserted cartilage particles; and/or the degree to which the cap member is demineralized, which determines the relative conformability of the cap member.

Moreover, in one aspect of the invention, the demineralized cancellous bone cap acts as a porous scaffold and provides sufficient structural support to withstand subsequent mechanical loading. In another embodiment, the addition of the cartilage particles to the demineralized cancellous bone cap further increases the stiffness of the region so as to provide adequate stiffness to withstand loading. In another aspect of the invention, the demineralized cancellous bone cap is sufficiently conformable (with or without the addition of cartilage particles) so as to be insertable into a tissue defect without significant damage to surrounding or opposing tissues. In an embodiment, the pliability of the demineralized cancellous bone prevents damage to surrounding or opposing cartilage surfaces during loading and articulation and allows the scaffold cap to conform to the natural curvature of the joint surface.

The cartilage particle gel or paste provides the environment and necessary biochemical cues to elicit a healing response from the cells that have infiltrated the construct from the surrounding host tissue, synovium and/or bleeding bone that undergoes blood clotting and other reparative processes. In an embodiment, these biochemical cues include the exposure to, or release of, various growth factors, as discussed herein.

The cartilage particles are preferably derived from allograft cartilage, such as allograft articular cartilage. For example, such cartilage particles may be composed at least partially of collagen type II and proteoglycans, which may provide a non-cellular matrix to support cell attachment and to facilitate extracellular matrix production. The cartilage particles may also be derived from fibrous cartilage, or a combination of hyaline and fibrous cartilage. Alternatively, autograft or xenograft cartilage may be used.

In an alternative embodiment, a gel or paste containing fibrous tissue particles (e.g., derived from meniscus, tendons, ligaments, annulus of an intervertebral disc, etc.) may be used for repairing defects in fibrous cartilage tissues (e.g., meniscus, tendons, ligaments, annulus of an intervertebral disc etc.) For example, defects in a meniscus may be repaired using a paste mixture containing cartilage particles derived from meniscus tissues.

In another embodiment, any of a number of tissues (e.g. meniscus, tendons, ligaments, skin, fascia, periosteum, muscle, fat, nucleous pulposus of intervertebral disc, etc.) may be freeze-milled and subsequently utilized in defect repair and/or genesis of similar or physiologically unrelated tissues.

In yet another embodiment, the matrix of the cartilage paste may comprise a plasma protein matrix. Such fibrin matrices are porous-structured, solid or semi-solid biodegradable substances having pores or spaces sufficiently large to allow cells to populate, or invade, the matrix. A polymerizing agent may be required to form the matrix, such as the addition of thrombin to a solution containing fibrinogen to form a fibrin matrix. The plasma protein matrices of the present invention may be used as a scaffold or as a sponge for culturing cells, as a tissue replacement implant, or as a cell-bearing tissue replacement implant. U.S. Pat. No. 7,009,039, issued Mar. 7, 2006, and U.S. Pat. No. 7,335,508, issued Feb. 26, 2008, the disclosures of both of which are incorporated by reference herein in their entireties, disclose elastic, freeze-dried (i.e., lyophilized) fibrin matrices useful for growing cells within the pores of the matrix.

In an embodiment of the invention, the aforesaid fibrin matrices comprise plasma proteins. In some embodiments of the invention, such plasma proteins may be any of the following: human plasma proteins, non-human mammalian plasma proteins, avian plasma protein, recombinant plasma proteins, engineered (i.e., synthetic or deliberately modified) plasma proteins, partially-purified plasma proteins, and totally-purified plasma proteins. In certain embodiments, such plasma proteins are allogeneous plasma proteins or a patient's autologous plasma proteins. In some embodiments, the fibrin matrix is obtained by mixing plasma proteins, including fibrinogen and Factor XIII, with thrombin and at least one anti-fibrolytic agent. In some embodiments, the freeze-dried fibrin matrix has a moisture content of less than 3%. In other embodiments, the freeze-dried fibrin matrix has a moisture content in the range of about 3% to about 9%, which allows easier and quicker implantation of the fibrin matrix with cartilage paste and growth factors. Such growth factors include, but are not limited to, FGF-2 and its variants and FGF-9 and its variants, and may include other growth factors discussed elsewhere herein.

The starting material from which cartilage particles are derived may be lyophilized. In an embodiment, the starting material from which cartilage particles are derived will have been lyophilized prior to freeze-milling, so that their water content may be within a range from about 0.1% to 8.0%. In another embodiment, the cartilage particles resulting from the freeze-milling process may be lyophilized again (i.e., re-lyophilized). In another embodiment, the cartilage particles resulting from the mincing or milling process may be rehydrated before re-lyophilization. In another embodiment, the cartilage particles resulting from the freeze-milling process may be inserted into a construct and relyophilized together with the construct.

The cartilage particles may range from about 10% to about 80% by weight of a gel or paste (in an embodiment, about 22%), and may be mixed with a biocompatible carrier, which constitutes the remaining weight of the gel or paste. The biocompatible carrier is preferably bioabsorbable. The carrier may have a composition that includes one or more of the following: phosphate buffered saline (PBS) solution, saline solution, sodium hyaluronate solution (HA) (molecular weight ranging from 7.0×10⁵ to 1.2×10⁶ Da), hyaluronic acid and its derivatives, fibrin, gelatin, collagen, chitosan, alginate, dextran, carboxymethylcellulose (CMC), hydroxypropyl methylcellulose, other polymers, blood and/or plasma.

The cartilage particles can be freeze-milled to have various particle sizes, and the carrier can have different viscosities, depending on the desired consistency of the gel or paste. The cartilage gel or paste can be deposited into the cap member, as described herein. The cartilage gel or paste enhances the tissue integration between the allograft construct and adjacent host tissue. For example, the use of cartilage gel or paste in repairing an articular cartilage defect may result in the production of new, well-organized articular cartilage tissue, accompanied by a restored “tidemark”.

A method of placing the cartilage defect repair material (i.e., the cartilage particle mixture disclosed herein, including a bioabsorbable carrier) in a cartilage defect site may include the steps of (a) cutting a patient's tissue to remove diseased cartilage from the cartilage defect site; (b) placing the cartilage particle mixture into the cartilage defect site; and (c) placing a cover over the placed mixture.

A method of repairing articular cartilage according to the present invention may include the step of placing a therapeutically effective amount of the cartilage defect repair material (i.e., the cartilage particle mixture disclosed herein, including a bioabsorbable carrier) into a cartilage defect site, wherein, subsequent to placement of the therapeutically effective amount of the cartilage defect repair material into the cartilage defect site, a greater percentage of repair tissue generated in the cartilage defect site is articular cartilage as compared to equivalent cartilage defect sites left untreated or treated with microfracture. The percentage of repair tissue generated may subsequently be assessed by relative uptake of Safranin-O and/or anti-collagen II staining materials by the repair tissue.

### Endogenous and Exogenous Growth Factors

As discussed above, cartilage paste or gel components are believed to provide the environmental and biochemical cues necessary to elicit a healing response from the cells. For example, cartilage that has been freeze-milled may have greater exposure/bioavailability of different endogenous cytokines, growth factors, etc. relative to the surrounding environment. These may include, without limitation, at least native FGF-2, IGF-1, TGF-β (including TGF-β1), BMP-2, and BMP-14 (GDF-5).

The cartilage particles may be provided alone or optionally packaged with a construct, and may be provided to a medical practitioner without added cells or added growth factors. Such cartilage particles (whether alone or in combination with a construct) are themselves capable of supporting articular cartilage regeneration without the addition of further materials.

As noted herein, cancellous bone constructs may also be loaded with one or more exogenous chondrogenic growth factor additives, including, but not limited to, recombinant or native or variant growth factors of FGF-2, FGF-4, FGF-5, FGF-7, FGF-9, FGF-11, FGF-21, TGF-β (including TGF-β1), BMP-2, BMP-4, BMP-7, BMP-14 (GDF-5), PDGF, VEGF, IGF-1, and bioreactive peptides such as Nell 1 (e.g., UCB1) and TP508. Additional growth factors which can be added include hepatocyte growth factor and platelet-derived growth factor. Other possible additives include human allogeneic or autologous chondrocytes, human allogeneic cells, human allogeneic or autologous bone marrow cells, human allogeneic or autologous stem cells, synovial cells, mesenchymal stem cells, pluripotent stem cells, mesenchymal stem and progenitor cells, stromal cells, cartilage stem cells, demineralized bone matrix, insulin, interleukin-1 receptor antagonist, Indian hedgehog, parathyroid hormone-related peptide, viral vectors for growth factor or DNA delivery, nanoparticles, platelet-rich plasma, fibrin clot, blood, bioabsorbable polymers, hyaluronic acid, bone marrow aspirate, xenogenic chondrocytes and mesenchymal stem cells, naked DNA, and RNA. Any one or more of the above-listed additives may be absorbed or combined with the constructs and/or the aforementioned cartilage-based paste and/or gel, or added directly to the cartilage particle mixtures described herein. As an illustration, a chondrogenic growth factor may be adsorbed into a construct, or into the cartilage particle gel or paste added to the construct, or into both the construct and the cartilage particle gel or paste.

In an embodiment, the growth factor TGF-β is included as an activatable endogenous component and/or as an exogenous component (latent or active) in any of the embodiments disclosed herein.

In another embodiment, any member of the growth factor family FGF, including FGF-2 and FGF-9, or a natural or recombinant variant thereof is included (as an endogenous component and/or as an exogenous component) in any of the embodiments disclosed herein. One description of a member of the FGF family's structure and physiological role (particularly relating to enhancing chondrogenesis and chondrogenesis lineage commitment from mesenchymal stem cells) is found in the article “FGF-2 Enhances the Mitotic and Chondrogenic Potentials of Human Adult Bone Marrow-Derived Mesenchymal Stem Cells” (L. A. Solchaga et al., Journal of Cellular Physiology 203:398-409 (2005)), which is incorporated herein by reference in its entirety. In an embodiment, FGF-2 binding enhances chondrocyte proliferation. In another embodiment, FGF-2 binding enhances chondrocyte differentiation. In another embodiment, FGF-2 binding increases chondrocyte aggregation. In another embodiment, FGF-2 binding increases development of chondrocyte-mediated creation of extracellular matrix. In another embodiment, FGF-2 binding increases proteoglycan synthesis. In another embodiment, FGF-2 binding mediates increased collagen type II/type I ratio as compared to control cells. In another embodiment, FGF-2 binding downregulates MAP kinase activities. In another embodiment, FGF-2 binding inhibits MAP kinase activities.

In another embodiment, freeze-milled cartilage particles having at least one dimension that is 212 microns or less, are combined with a phosphate buffered saline carrier and an exogenous fibroblast growth factor such as FGF-2 or a variant thereof, or FGF-9 or a variant thereof, in a therapeutically effective and/or efficacious dosage. This combination may be infused into a construct using the protocol outlined above. In another embodiment, the freeze-milled cartilage particles preferably have at least one dimension within a range of from approximately 10 microns to approximately 212 microns.

In one example, a fibroblast growth factor variant FGF-2v, (Pro-Chon Biotech, Rehovot, Israel) as described in U.S. Pat. No. 7,563,769, issued Jul. 21, 2009, or International (PCT) Publication No. WO 2008/038287, published Apr. 3, 2008, both of which are incorporated by reference herein in their entirety, may be utilized. In an embodiment, the dosage of FGF-2v ranges from 0.5-5,000 micrograms/ml of cartilage particle mixture.

In an embodiment, an FGF-2 or FGF-9 variant may be characterized relative to wild-type FGF-2 or FGF-9 as having at least one of the following attributes: enhanced specificity for one receptor subtype, increased biological activity mediated by at least one receptor subtype with equivalent or reduced activity mediated through another receptor subtype; enhanced affinity to at least one receptor subtype resulting in increased cell proliferation and differentiation mediated through that receptor subtype.

In another embodiment, the use of an FGF-2 variant in conjunction with cartilage particles may result in accelerated cartilage repair resulting in better organized and/or more mature replacement articular cartilage, accompanied by formation of a restored “tidemark”; and a time release profile (e.g., slow and/or consistent release) of bound FGF-2v from cartilage particles, as discussed further herein in Example 13. FGF-2 and its variants are discussed more fully elsewhere herein.

In another embodiment, any member of the growth factor family BMP is included (as an endogenous component and/or as an exogenous component) in any of the embodiments disclosed herein. One description of a member of the BMP family's structure and physiological role (particularly relating to initiating chondrogenesis and chondrogenesis lineage commitment from mesenchymal stem cells) is found in the article “BMP2 initiates chondrogenic lineage development of adult human mesenchymal stem cells in high-density culture” (B. Schmitt et al., Differentiation (2003) 71:567-577), incorporated herein by reference in its entirety. In an embodiment, BMP2 may be co-administered with TGF-β3 so as to drive chondrocyte differentiation from MSCs (mesenchymal stem cells). In another embodiment, BMP2 may drive selective differentiation. In another embodiment, administration of BMP2 results in substantially no adipocyte or osteoclast cell differentiation. In another embodiment, BMP2 facilitates upregulation of COMP-type II collagen and cartilage oligomeric matrix protein synthesis. In another embodiment, BMP2 facilitates development of high density chondrocyte microenvironments, which may be important for cell-to-cell signaling so as to maintain chondrocyte lineage.

Fibroblast Growth Factors (FGF) and their Variants

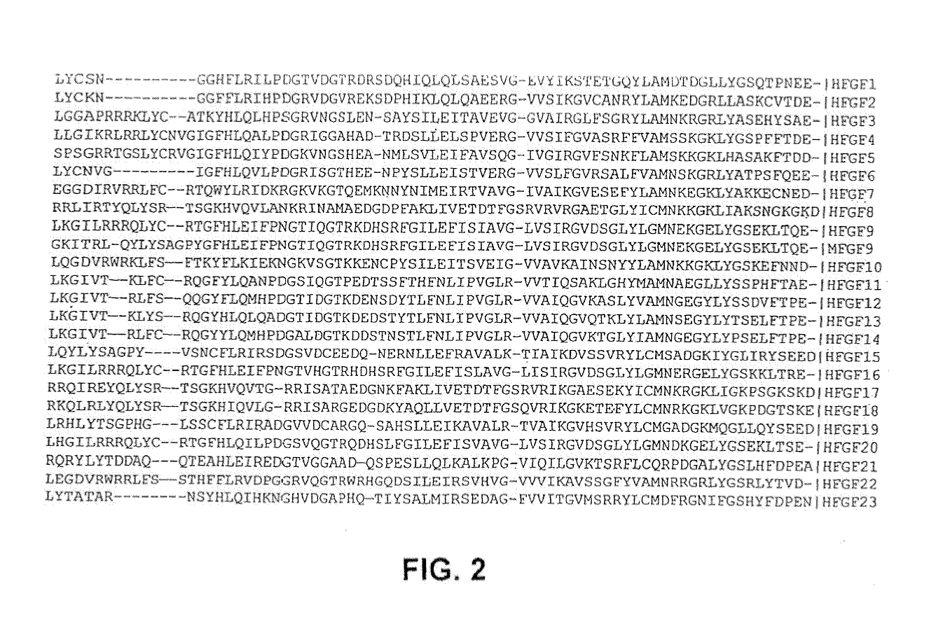

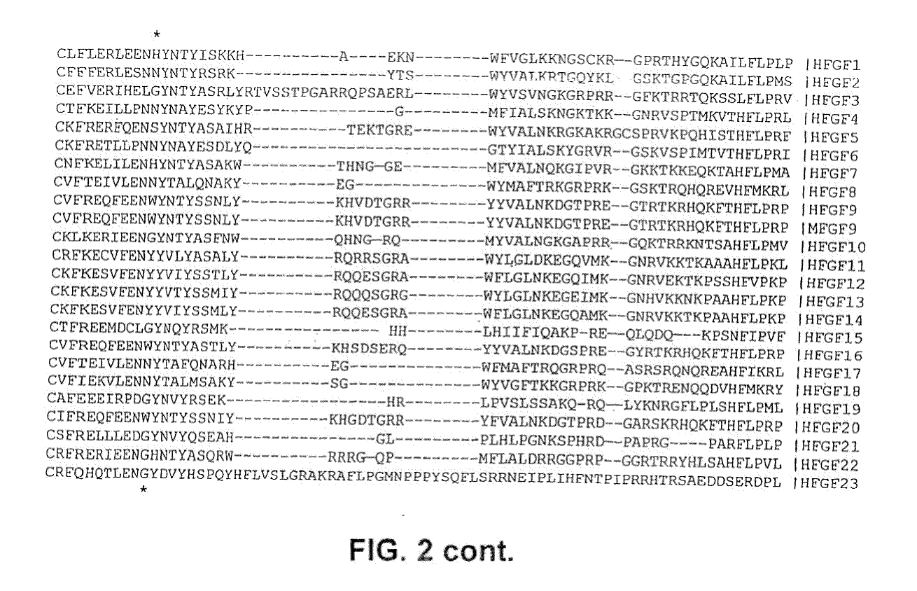

Fibroblast growth factors (FGFs) constitute a large family of structurally related, heparin binding polypeptides, which are expressed in a wide variety of cells and tissues. Overall, the FGFs share between 17-72% amino acid sequence homology and a high level of structural similarity. A homology core of around 120 amino acids is highly conserved and has been identified in all members of the family. The residues of the core domain interact with both a fibroblast growth factor receptor (FGFR) and heparin. Twelve antiparallel B strands, referred to as β1 through β12, have been identified in the core structure. The strands are linked one to another by loops of variable lengths that are organized into a trefoil internal symmetry. The amino acid sequence of the core structure of the known FGFs is depicted herein in FIG. 2.

As disclosed in the aforementioned U.S. Pat. No. 7,563,769, FGF variants, including variants of FGF-2 and FGF-9, may have altered binding to a receptor compared to that of the native parent FGF, altering the specificity of the variant for one or more receptors. Accordingly, as also disclosed in U.S. Pat. No. 7,563,769, FGF variants comprising amino acid substitutions in the loop between the β8 and β9 strands of the core structure (hereinafter, “the β8-β9 loop”) may have improved properties over native FGF, in addition to altered specificity to FGFRs. In certain embodiments the amino acid substitution yields variants with superagonist properties. Certain amino acid substitutions in the β8-β9 loop yield polypeptides with improved properties over the wild type FGF, including high binding affinity, reduced biological activity and enhanced receptor specificity, thus providing therapeutically beneficial molecules for stimulating growth of chondrocytes. Descriptions of various FGFRs, including FGFR1, FGFR2, FGFR3, FGFR3IIIb and FGFR3IIIc are available in the prior art and may readily be discovered through routine searches of the relevant literature.

As disclosed in International (PCT) Publication WO 02/36732, published May 10, 2002, the disclosure of which is incorporated by reference herein in its entirety, FGF variants comprising mutations in the β8-β9 loop provide enhanced receptor subtype specificity. The aforesaid U.S. Pat. No. 7,563,769 discloses increased receptor specificity and/or affinity and enhanced biological activity of FGF ligands by amino acid substitutions in the β8-β9 loop, specifically at position N111 of wild type FGF-2 or positions N143 or W144 of wild type FGF-9. The amino acid (“aa”) numbering of FGF-2 used herein is according to the 155 aa isoform; amino acid 107 would be amino acid 98 in the 146 aa isoform. FGF-2 variants wherein asparagine at position 111 (N111) is substituted with another residue unexpectedly exhibit both an increase in biological activity and increased receptor specificity.

An FGF-2 variant according to an embodiment of the invention is denoted FGF2-N111X wherein X is an amino acid other than asparagine. According to certain embodiments of the invention X is selected from among glycine (Gly, G) and arginine (Arg, R). This sequence of this variant is denoted herein as FGF2-N111X, SEQ ID NO: 1. Another embodiment of the present invention provides a variant of FGF-2, denoted as herein FGF2-N111R, having SEQ ID NO: 2, wherein substitution of the asparagine 111 with arginine (Arg, R) shows essentially unchanged activity towards FGFR3 and FGFR2 while increasing activity for FGFR1. Yet another embodiment of the present invention provides a variant of FGF-2, denoted herein as FGF2-N111G, having SEQ ID NO: 3, wherein substitution of the asparagine 111 with glycine (Gly, G) shows essentially unchanged activity towards FGFR3 while increasing activity for FGFR1, and to a lesser extent towards FGFR2. It may be noted that disclosures made in the aforementioned U.S. Pat. No. 7,563,769 (e.g., in Example 11) show that FGF variant FGF2-N111G is more effective in proliferating articular chondrocytes than the FGF-2 wild type or other FGF in vitro. This finding suggests that this variant may also be particularly effective in promoting growth of articular chondrocytes in vivo.

Other FGF-2 variants of the invention may further comprise additional modifications within, or outside of, the β8-β9 loop. Examples of modifications include truncations of the N- or C-terminus or both termini and/or amino acid substitutions, deletions or additions wherein the variants retain superior mitogenic activity mediated via FGFRs with unimpaired or improved affinities compared to the wild type parent FGF-2, from which the variant was derived. The additional modifications function to improve certain properties of the variants including enhanced stability, increased yield of recombinants, solubility and other properties known in the art. For example, FGF-2 may comprise amino acid substitutions at amino acid positions 3 and 5 wherein alanine (Ala, A) and serine (Ser, S) are replaced with glutamine (Gln, Q) (A3Q and S5Q) providing variants with improved yields and stability. An embodiment of the present invention provides such an FGF-2 variant, denoted herein as FGF2(3,5Q)-N111X, having SEQ ID NO: 4, where X denotes an asparagine substitution by arginine (Arg, R) or glycine (Gly, G). The FGF-2 variant having the asparagine at position 111 replaced by glycine, and the alanine at position 3 and the serine at position 5 replaced by glutamine, is denoted herein as FGF2(3,5Q)-N111G, having SEQ ID NO: 5, and referred to, hereinafter, as “FGF2v1”. FGF2v1 shows essentially unchanged activity towards FGFR3IIIb and FGFR2 while increasing activity for FGFR1 and FGFR3IIIc.

A variant of FGF-9 according to the present invention has a 63 amino acid N-terminus truncation, and is denoted herein as R64M-FGF9. Another variant of FGF-9 according to the present invention has a 63 amino acid N-terminus truncation and an 18 amino acid C-terminus truncation, and is denoted herein as FGF9-2. Both the R64M-FGF9 variant and the FGF9-2 variant have the amino acid tryptophan at position 144 (W144) of the β8-β9 loop, which is the positional equivalent of the N111 position of FGF-2. Further variants of R64M-FGF9 and FGF9-2 have substitutions at W144, denoted herein as R64M-FGF9-W144X, having SEQ ID NO: 6 and FGF9-2-W144X, having SEQ ID NO: 7, respectively. According to certain embodiments of the invention, X is selected from among glycine (Gly, G), arginine (Arg, R), glutamate (Glu, E) and valine (Val, V). Such R64M-FGF9-W114X variants and FGF9-2-W144X variants abolish the binding to FGFR1, while retaining high affinity binding to FGFR3 and a lesser affinity to FGFR2. It may be noted that disclosures made in the aforementioned U.S. Pat. No. 7,563,769 (e.g., in Example 12) indicate that the glycine-substituted variant enhances differentiation of articular chondrocytes.

Other variants of R64M-FGF9 and FGF9-2 have substitutions at the asparagine 143 (N143) position of the β8-β9 loop, rather than at the W144 position, and are denoted herein as R64M-FGF9-N143X, SEQ ID NO: 8 and FGF-2-N143X, SEQ ID NO: 9, respectively. In such variants, X may be selected from among amino acids other than asparagine, including, but not limited to, serine (Ser, S), such that the variant abolishes the binding to FGFR1, while retaining high affinity binding to FGFR3 and a lesser affinity to FGFR2.

### Methods of Producing FGF Variants

One of the available methods for producing FGF variants is through recombinant DNA technologies, well known to those skilled in the art. For example, the variants may be prepared by polymerase chain reaction (PCR) using specific primers for each of the truncated forms or the amino acid substitutions that are disclosed hereinbelow. As used herein, the term “polymerase chain reaction” (“PCR”) refers to the methods disclosed in U.S. Pat. Nos. 4,683,195; 4,683,202 and 4,965,188, which are hereby incorporated by reference in their entirety. The PCR fragments may be purified on an agarose gel and the purified DNA fragment may be cloned into an expression vector and transfected into host cells. The host cells may be cultured and the protein harvested according to methods known in the art. Alternatively, FGF variants may be produced by other methods of producing polypeptides or proteins known to those skilled in the art.

### FGF-2 Variant Sequences

The amino acid sequences of FGF-2 variants used in certain embodiments of the present invention are disclosed as follows:

(i) Amino Acid Sequence of FGF2-N111X
(SEQ ID NO: 1)

MAAGSITTLP ALPEDGGSGA FPPGHFKDPK RLYCKNGGFF
LRIHPDGRVD GVREKSDPHI KLQLQAEERG VVSIKGVCAN
RYLAMKEDGR LLASKCVTDE CFFFERLESN XYNTYRSRKY
TSWYVALKRT GQYKLGSKTG PGQKAILFLP MSAKS

wherein X is an amino acid other than N, such as, but not limited to, R or G;

(ii) Amino Acid Sequence of FGF2-N111R

(SEQ ID NO: 2)

MAAGSITTLP ALPEDGGSGA FPPGHFKDPK RLYCKNGGFF
LRIHPDGRVD GVREKSDPHI KLQLQAEERG VVSIKGVCAN
RYLAMKEDGR LLASKCVTDE CFFFERLESN RYNTYRSRKY
TSWYVALKRT GQYKLGSKTG PGQKAILFLP MSAKS;
(iii) Amino Acid Sequence of FGF2-N111G

(SEQ ID NO: 3)

MAAGSITTLP ALPEDGGSGA FPPGHFKDPK RLYCKNGGFF
LRIHPDGRVD GVREKSDPHI KLQLQAEERG VVSIKGVCAN
RYLAMKEDGR LLASKCVTDE CFFFERLESN GYNTYRSRKY
TSWYVALKRT GQYKLGSKTG PGQKAILFLP MSAKS; and
(iv) Amino Acid Sequence of FGF2(3,5Q)-N111X

(SEQ ID NO: 4)

MAQGQITTLP ALPEDGGSGA FPPGHFKDPK RLYCKNGGFF
LRIHPDGRVD GVREKSDPHI KLQLQAEERG VVSIKGVCAN
RYLAMKEDGR LLASKCVTDE CFFFERLESN XYNTYRSRKY
TSWYVALKRT GQYKLGSKTG PGQKAILFLP MSAKS

wherein X is an amino acid other than N, such as, but not limited to, R or G.

(v) Amino Acid Sequence of FGF2(3,5Q)-N111G

(SEQ ID NO: 5)

MAQGQITTLP ALPEDGGSGA FPPGHFKDPK RLYCKNGGFF
LRIHPDGRVD GVREKSDPHI KLQLQAEERG VVSIKGVCAN
RYLAMKEDGR LLASKCVTDE CFFFERLESN GYNTYRSRKY
TSWYVALKRT GQYKLGSKTG PGQKAILFLP MSAKS

The amino acid sequences of FGF-9 variants used in certain embodiments of the present invention are disclosed as follows:

(vi) Amino Acid sequence of R64M-FGF9-W144X 145 aa

(SEQ ID NO: 6)

MQLYCRTGFH LEIFPNGTIQ GTRKDHSRFG ILEFISIAVG
LVSIRGVDSG LYLGMNEKGE LYGSEKLTQE CVFREQFEEN
XYNTYSSNLY KHVDTGRRYY VALNKDGTPR EGTRTKRHQK
FTHFLPRPVD PDKVPELYKD ILSQS

wherein X is other than W and more preferably selected from G, R, E or V.

(vii) Amino Acid sequence of FGF9-2-W144X 127 aa

(SEQ ID NO: 7)

MQLYCRTGFH LEIFPNGTIQ GTRKDHSRFG ILEFISIAVG
LVSIRGVDSG LYLGMNEKGE LYGSEKLTQE CVFREQFEEN
XYNTYSSNLY KHVDTGRRYY VALNKDGTPR EGTRTKRHQK
FTHFLPR

wherein X is other than W and more preferably selected from G, R, E or V.

(viii) Amino Acid sequence of R64M-FGF9-N143X
145 aa

(SEQ ID NO: 8)

MQLYCRTGFH LEIFPNGTIQ GTRKDHSRFG ILEFISIAVG
LVSIRGVDSG LYLGMNEKGE LYGSEKLTQE CVFREQFEEX
WYNTYSSNLY KHVDTGRRYY VALNKDGTPR EGTRTKRHQK
FTHFLPRPVD PDKVPELYKD ILSQS

wherein X is other than N and more preferably S.

(ix) Amino Acid sequence of FGF9-2-N143X 127 aa

(SEQ ID NO: 9)

MQLYCRTGFH LEIFPNGTIQ GTRKDNSRFG ILEFISIAVG
LVSIRGVDSG LYLGMNEKGE LYGSEKLTQE CVFREQFEEX
WYNTYSSNLY KHVDTGRRYY VALNKDGTPR EGTRTKRHQK
FTHFLPR

wherein X is other than N and more preferably S.

The polynucleotide sequences corresponding to the amino acid sequences of FGF-2 variants related above are disclosed as follows:

(i) Sequence of FGF2-N111X DNA

(SEQ ID NO: 10)

ATGGCTGCCG GGAGCATCAC CACGCTGCCC GCCCTTCCGG
AGGATGGCGG CAGCGGCGCC TTCCCGCCCG GGCACTTCAA
GGACCCCAAG CGGCTGTACT GCAAAAACGG GGGCTTCTTC
CTGCGCATCC ACCCCGACGG CCGAGTTGAC GGGGTCCGGG
AGAAGAGCGA CCCTCACATC AAGCTACAAC TTCAAGCAGA
AGAGAGAGGA GTTGTGTCTA TCAAAGGAGT GTGTGCTAAC
CGGTACCTGG CTATGAAGGA AGATGGAAGA TTACTGGCTT
CTAAATGTGT TACGGATGAG TGTTTCTTTT TTGAACGATT
GGAATCTAAT NNNTACAATA CTTACCGGTC TAGAAAATAC
ACCAGTTGGT ATGTGGCATT GAAACGAACT GGGCAGTATA
AACTTGGTTC CAAAACAGGA CCTGGGCAGA AAGCTATACT
TTTTCTTCCA ATGTCTGCTA AGAGCTGA

wherein NNN is other than a codon coding for Asn (AAT or AAC) or a stop codon, and is more preferably a codon coding for amino acid Gly or Arg;

(ii) Sequence of FGF2-N111G DNA

(SEQ ID NO: 11)

ATGGCTGCCG GGAGCATCAC CACGCTGCCC GCCCTTCCGG
AGGATGGCGG CAGCGGCGCC TTCCCGCCCG GGCACTTCAA
GGACCCCAAG CGGCTGTACT GCAAAAACGG GGGCTTCTTC
CTGCGCATCC ACCCCGACGG CCGAGTTGAC GGGGTCCGGG
AGAAGAGCGA CCCTCACATC AAGCTACAAC TTCAAGCAGA
AGAGAGAGGA GTTGTGTCTA TCAAAGGAGT GTGTGCTAAC
CGGTACCTGG CTATGAAGGA AGATGGAAGA TTACTGGCTT
CTAAATGTGT TACGGATGAG TGTTTCTTTT TTGAACGATT
GGAATCTAAT NNNTACAATA CTTACCGGTC TAGAAAATAC
ACCAGTTGGT ATGTGGCATT GAAACGAACT GGGCAGTATA
AACTTGGTTC CAAAACAGGA CCTGGGCAGA AAGCTATACT
TTTTCTTCCA ATGTCTGCTA AGAGCTGA

wherein NNN is a codon coding for amino acid Gly (GGT, GGC, GGA, GGG);

(iii) Sequence of FGF2-N111R DNA

(SEQ ID NO: 12)

ATGGCTGCCG GGAGCATCAC CACGCTGCCC GCCCTTCCGG
AGGATGGCGG CAGCGGCGCC TTCCCGCCCG GGCACTTCAA
GGACCCCAAG CGGCTGTACT GCAAAAACGG GGGCTTCTTC
CTGCGCATCC ACCCCGACGG CCGAGTTGAC GGGGTCCGGG
AGAAGAGCGA CCCTCACATC AAGCTACAAC TTCAAGCAGA
AGAGAGAGGA GTTGTGTCTA TCAAAGGAGT GTGTGCTAAC
CGGTACCTGG CTATGAAGGA AGATGGAAGA TTACTGGCTT
CTAAATGTGT TACGGATGAG TGTTTCTTTT TTGAACGATT
GGAATCTAAT CGNTACAATA CTTACCGGTC TAGAAAATAC
ACCAGTTGGT ATGTGGCATT GAAACGAACT GGGCAGTATA
AACTTGGTTC CAAAACAGGA CCTGGGCAGA AAGCTATACT
TTTTCTTCCA ATGTCTGCTA AGAGCTGA

wherein N is a nucleotide selected from A, C, G or T; and

(iv) Sequence of FGF2(3Q5Q)-N111X DNA

(SEQ ID NO: 13)

ATGGCTCAXG GGCAXATCAC CACGCTGCCC GCCCTTCCGG
AGGATGGCGG CAGCGGCGCC TTCCCGCCCG GGCACTTCAA
GGACCCCAAG CGGCTGTACT GCAAAAACGG GGGCTTCTTC
CTGCGCATCC ACCCCGACGG CCGAGTTGAC GGGGTCCGGG
AGAAGAGCGA CCCTCACATC AAGCTACAAC TTCAAGCAGA
AGAGAGAGGA GTTGTGTCTA TCAAAGGAGT GTGTGCTAAC
CGGTACCTGG CTATGAAGGA AGATGGAAGA TTACTGGCTT
CTAAATGTGT TACGGATGAG TGTTTCTTTT TTGAACGATT
GGAATCTAAT NNNTACAATA CTTACCGGTC TAGAAAATAC
ACCAGTTGGT ATGTGGCATT GAAACGAACT GGGCAGTATA
AACTTGGTTC CAAAACAGGA CCTGGGCAGA AAGCTATACT
TTTTCTTCCA ATGTCTGCTA AGAGCTGA

wherein nucleotides 9 and 15 are independently chosen from A or G and the codon encoded by NNN at position 331-333 is other than a codon coding for Asn (AAT or AAC) or a stop codon and, more preferably, encodes for amino acid Gly or Arg.

(v) Sequence of FGF2(3Q5Q)-N111G DNA

(SEQ ID NO: 14)

ATGGCTCAXG GGCAXATCAC CACGCTGCCC GCCCTTCCGG
AGGATGGCGG CAGCGGCGCC TTCCCGCCCG GGCACTTCAA
GGACCCCAAG CGGCTGTACT GCAAAAACGG GGGCTTCTTC
CTGCGCATCC ACCCCGACGG CCGAGTTGAC GGGGTCCGGG
AGAAGAGCGA CCCTCACATC AAGCTACAAC TTCAAGCAGA
AGAGAGAGGA GTTGTGTCTA TCAAAGGAGT GTGTGCTAAC
CGGTACCTGG CTATGAAGGA AGATGGAAGA TTACTGGCTT
CTAAATGTGT TACGGATGAG TGTTTCTTTT TTGAACGATT
GGAATCTAAT GGNTACAATA CTTACCGGTC TAGAAAATAC
ACCAGTTGGT ATGTGGCATT GAAACGAACT GGGCAGTATA
AACTTGGTTC CAAAACAGGA CCTGGGCAGA AAGCTATACT
TTTTCTTCCA ATGTCTGCTA AGAGCTGA

wherein nucleotides 9 and 15 are independently chosen from A or G and the N at position 333 is selected from A, C, G or T.

The polynucleotide sequences corresponding to the amino acid sequences of FGF-9 variants related above are disclosed as follows:

(vi) Sequence of R64M-FGF9-W144X DNA

(SEQ ID NO: 15)

A
TGCAGCTATA CTGCAGGACT GGATTTCACT TAGAAATCTT
CCCCAATGGT ACTATCCAGG GAACCAGGAA AGACCACAGC
CGATTTGGCA TTCTGGAATT TATCAGTATA GCAGTGGGCC
TGGTCAGCAT TCGAGGCGTG GACAGTGGAC TCTACCTCGG
GATGAATGAG AAGGGGGAGC TGTATGGATC AGAAAAACTA
ACCCAAGAGT GTGTATTCAG AGAACAGTTC GAAGAAAACN
NNTATAATAC GTACTCGTCA AACCTATATA AGCACGTGGA
CACTGGAAGG CGATACTATG TTGCATTAAA TAAAGATGGG
ACCCCGAGAG AAGGGACTAG GACTAAACGG CACCAGAAAT
TCACACATTT TTTACCTAGA CCAGTGGACC CCGACAAAGT
ACCTGAACTG TATAAGGATA TTCTAAGCCA AAGTTGA

wherein NNN is other than a codon coding for Trp (TGG) or a stop codon (TAA, TAG or TGA) and is more preferably a codon coding for amino acid Gly, Arg, Val or Glu.

(vii) Sequence of FGF9-2-W144X DNA

(SEQ ID NO: 16)

A
TGCAGCTATA CTGCAGGACT GGATTTCACT TAGAAATCTT
CCCCAATGGT ACTATCCAGG GAACCAGGAA AGACCACAGC
CGATTTGGCA TTCTGGAATT TATCAGTATA GCAGTGGGCC
TGGTCAGCAT TCGAGGCGTG GACAGTGGAC TCTACCTCGG
GATGAATGAG AAGGGGGAGC TGTATGGATC AGAAAAACTA
ACCCAAGAGT GTGTATTCAG AGAACAGTTC GAAGAAAACN
NNTATAATAC GTACTCGTCA AACCTATATA AGCACGTGGA
CACTGGAAGG CGATACTATG TTGCATTAAA TAAAGATGGG
ACCCCGAGAG AAGGGACTAG GACTAAACGG CACCAGAAAT
TCACACATTT TTTACCTAGA TGA

wherein NNN is other than a codon coding for Trp (TGG) or a stop codon (TAA, TAG or TGA) and is more preferably a codon coding for amino acid Gly, Arg, Val or Glu.

(viii) Sequence of R64M-FGF9-N143X DNA

(SEQ ID NO: 17)

A
TGCAGCTATA CTGCAGGACT GGATTTCACT TAGAAATCTT
CCCCAATGGT ACTATCCAGG GAACCAGGAA AGACCACAGC
CGATTTGGCA TTCTGGAATT TATCAGTATA GCAGTGGGCC
TGGTCAGCAT TCGAGGCGTG GACAGTGGAC TCTACCTCGG
GATGAATGAG AAGGGGGAGC TGTATGGATC AGAAAAACTA
ACCCAAGAGT GTGTATTCAG AGAACAGTTC GAAGAANNNT
GGTATAATAC GTACTCGTCA AACCTATATA AGCACGTGGA
CACTGGAAGG CGATACTATG TTGCATTAAA TAAAGATGGG
ACCCCGAGAG AAGGGACTAG GACTAAACGG CACCAGAAAT
TCACACATTT TTTACCTAGA CCAGTGGACC CCGACAAAGT
ACCTGAACTG TATAAGGATA TTCTAAGCCA AAGTTGA

wherein NNN is other than a codon coding for Asn (AAT, AAC) or a stop codon (TAA, TAG or TGA) and is more preferably a codon coding for amino acid Ser.

(ix) Sequence of FGF9-2-N143X DNA

(SEQ ID NO: 18)

A
TGCAGCTATA CTGCAGGACT GGATTTCACT TAGAAATCTT
CCCCAATGGT ACTATCCAGG GAACCAGGAA AGACCACAGC
CGATTTGGCA TTCTGGAATT TATCAGTATA GCAGTGGGCC
TGGTCAGCAT TCGAGGCGTG GACAGTGGAC TCTACCTCGG
GATGAATGAG AAGGGGGAGC TGTATGGATC AGAAAAACTA
ACCCAAGAGT GTGTATTCAG AGAACAGTTC GAAGAANNNT
GGTATAATAC GTACTCGTCA AACCTATATA AGCACGTGGA
CACTGGAAGG CGATACTATG TTGCATTAAA TAAAGATGGG
ACCCCGAGAG AAGGGACTAG GACTAAACGG CACCAGAAAT
TCACACATTT TTTACCTAGA TGA

wherein NNN is other than a codon coding for Asn (AAT, AAC) or a stop codon (TAA, TAG or TGA) and is more preferably a codon coding for amino acid Ser.

### Activation of Latent Endogenous Growth Factors

In an embodiment, the small size of the cartilage particles may facilitate increased activation of various latent forms of growth factors due to the increased aggregate and/or accessible surface area of the cartilage particles used. Examples specific to TGF-β are herein described, but the mechanical, physical and/or chemical activation processes described herein are applicable to a wide range of latent endogenous growth factors.

TGF-β is synthesized and secreted as a biologically inactive or “latent” complex. Activation must occur to release the mature, biologically active, form of TGF-β, for signal transduction. The mechanism of activation of latent TGF-β in vivo is not completely understood. It may occur by local acidification at the site of action or by endogenous and/or exogenous enzymatic activity, and may also involve integrins, thrombospondin, metalloproteases, plasmin, furin and other proteases. Latent TGF-β (L-TGF-β) can be activated in vitro by acid or alkaline solutions (pH 2 or pH 8, respectively), exposure to heat (e.g., 100° C.), or by treatment with chaotropic agents and substances like sodium dodecyl sulfate (SDS) and urea. In an embodiment, the molecular weight of TGF-(3 is reduced from 100 kDa to 25 kDa prior to or simultaneously with activation.

Various physiological substances have been reported to activate L-TGF-β in in vitro studies. Some examples are serine protease, plasmin, other proteases such as endoglycosidase F, sialidase, neuraminidase, cathepsins B and D, calpain, and the glycoprotein, thrombospondin-1, all of which can convert L-TGF-β to biologically active TGF-β. In an embodiment, TGF-β1 is cleaved from the C-terminus of a disulfide-linked dimer of pro-TGF-β1 by a subtilsin-like pro-protein convertase protease. It is normally secreted as an inactive, or latent, complex. Although it is not always stated, the isoform most often described to be susceptible to the actions of the aforementioned substances is TGF-β1.

Increased exposure, release, or activation of various growth factors may also be attributable to pH-mediated physical and/or chemical changes to the tissue. In another embodiment, such pH-mediated physical and/or chemical changes resulting in exposure, release, or activation of various growth factors are attributable to an acidic pH (for example, pH 2). In another embodiment, such pH-mediated physical and/or chemical changes resulting in exposure, release, or activation of various growth factors is attributable to an alkaline pH (for example, pH 8).

Increased exposure, release, or activation of various growth factors may also be attributable to temperature-mediated physical and/or chemical changes to the tissue. In an embodiment, growth factor activation occurs at mammalian body temperature (e.g., 37° C.). In another embodiment, growth factor activation is inhibited at low temperatures (e.g., −40° C.) with a subsequent measurable increase in growth factor structural stability. In an embodiment, the physiological mechanism of release from latency is an important control for the regulation and localization of TGF-β activity. In another embodiment, proteolysis of latent TGF-β is likely a part of the mechanism of release from latency.

Increased exposure, release, or activation of various growth factors may also be attributable to release of endogenous proteases and subsequent protease-mediated physical and/or chemical changes to the tissue. In an embodiment, the endogenous protease is serine protease. In another embodiment, the endogenous protease is a cathepsin. In another embodiment, the endogenous protease is a sialidase. In another embodiment, the sialidase is a neuramidase. In another embodiment, the endogenous protease is an endoglycosidase. In another embodiment, the endoglycosidase is endoglycosidase F, retinoic acid, and/or transglutaminase.

Increased exposure, release, or activation of various growth factors may also be attributable to release of chaotropic agents and subsequent physical and/or chemical changes to the tissue.

Increased exposure, release, or activation of various endogenous growth factors may also be attributable to the mechanical disruption of the freeze-milled cartilage. In another embodiment, increased exposure, release, and/or activation of various growth factors is attributable to the mechanical disruption of the freeze-milled cartilage, resulting in increased exposure of cartilage proteoglycans and other cartilage components to the outside environment.

Increased exposure, release, or activation of various endogenous growth factors may also be attributable to lyophilization of the freeze-milled cartilage, either before or after freeze-milling.

Increased exposure, release, or activation of various endogenous growth factors may also be attributable to conversion of one or more other growth factors from the latent stage.

Growth factor effects may be context-dependent; e.g. a growth factor that would drive osteogenesis in a vascularized environment will drive chondrogenesis in an avascular environment.

In an embodiment, the growth factor isoform often found to be susceptible to the actions of the aforementioned substances and/or manipulations is latent TGF-β1. In another embodiment, the growth factor isoforms often found to be susceptible to the actions of the aforementioned substances and/or manipulations are L-TGF-β2 and L-TGF-β3.

### Activation of Exogenous Growth Factors

The cartilage particle gel or paste can also contain exogenous growth factors and/or growth factor activators. The levels of these growth factors may be similar to or greater than the levels of endogenous growth factors in intact cartilage. Exogenous growth factors and/or growth factor activators can also be combined with the cartilage particles. In an embodiment, the cartilage particles are mixed with a growth factor in an aqueous vehicle, lyophilized and stored dry at room temperature. The cartilage particles with growth factors may, alternatively, be frozen. Alternatively, the mixture of cartilage particles and growth factors may be used immediately. In an embodiment, particles containing chondrogenic growth factors can be added to any portion of a construct according to the present invention, and particles containing osteogenic growth factors can be added to any portion of the construct except for the demineralized cancellous cap member. In an embodiment, the mixture containing the cartilage particles and growth factor can be lyophilized for storage. In an embodiment, the lyophilized cartilage particles and growth factor may have a residual water content that is within a range of from 0.1% to 8.0% by weight.

In another embodiment, the activatable exogenous growth factor can be any one of a variety of growth factors known to promote wound healing, cartilage and/or bone development (e.g., TGF-β).

In another embodiment, the activating agent used to solubilize the growth factor and/or adsorb it into the cartilage particles (or alternately to activate endogenous growth factors present in the freeze-milled cartilage particles) can be saline, water, PBS, Ringers, any agent capable of pH modification or proteolytic activity, etc.

In another embodiment, the resulting enhanced cartilage particles can contain levels of growth factors that are higher than the levels found in intact cartilage. In another embodiment, the cartilage particle mixture can be infused into all or part of the construct. If desired, the cartilage particle mixture can be infused primarily into a demineralized portion of the construct.

In another embodiment, cells which have been collected from the patient or grown outside of the patient can be inserted into the entire construct or into a demineralized portion (e.g., a cap member) thereof before, during or after deposit of the construct into the defect area. Such cells include, for example, allogenic or autologous bone marrow cells, stem cells and chondrocyte cells. A therapeutically effective cellular density may be utilized. In an embodiment, the cellular density of the cells is preferably within a range of from 1.0×10⁸ to 5.0×10⁸ cells/ml of paste or gel mixture. In another embodiment, the cellular density of the cells is preferably within a range of from 5.0×10⁶ to 1.0×10⁸ cells/ml of paste or gel mixture.

In another embodiment, any of the methods of the instant invention can be utilized to repair or stimulate growth of meniscus, muscle, tendons, ligaments, skin, periosteum and fat tissue. In another embodiment, meniscus, muscle, tendons, ligaments, skin, periosteum and/or fat tissue may itself be particularized and subsequently utilized to repair analogous and/or nonanalogous tissues.

The following examples are put forth, together with the disclosures made elsewhere herein, so as to provide those of ordinary skill in the art with a complete disclosure and description of how to make and use the present invention, and are not intended to limit the scope of what the inventors regard as their invention nor are they intended to represent that the experiments described below are all or the only experiments performed. Efforts have been made to ensure accuracy with respect to the numbers used (e.g., amounts, temperature, etc.), but some experimental errors and deviations should be accounted for. Unless indicated otherwise, parts are parts by weight, molecular weight is weight average molecular weight, temperature is in degrees Celsius (e.g., “° C.”), and pressure is at or near atmospheric.

### Example 1

### Measurement of Demineralized Construct Porosity

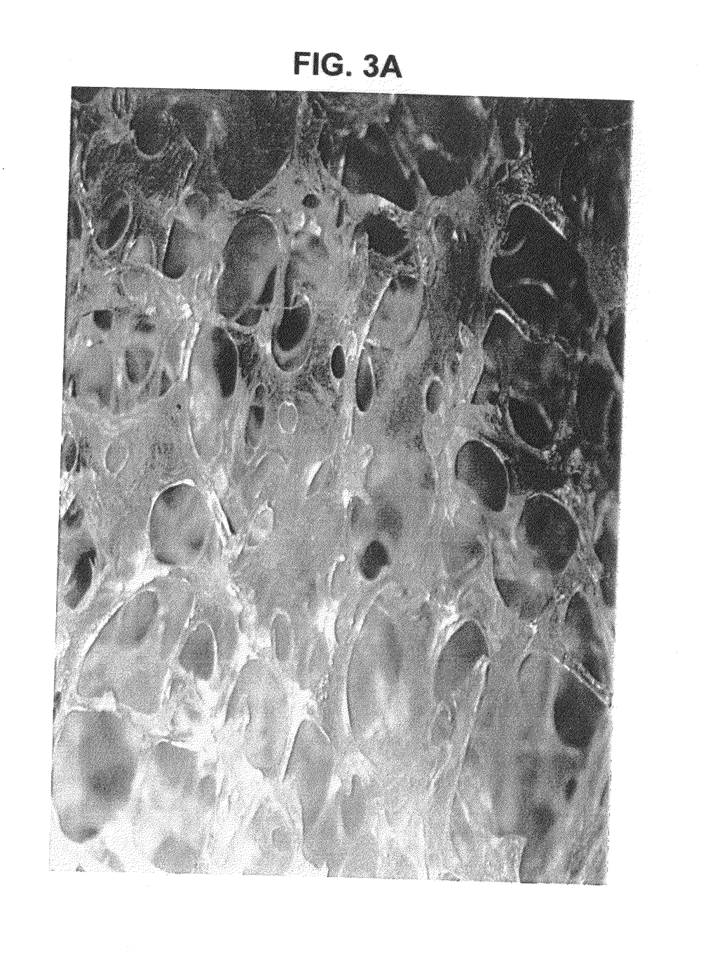

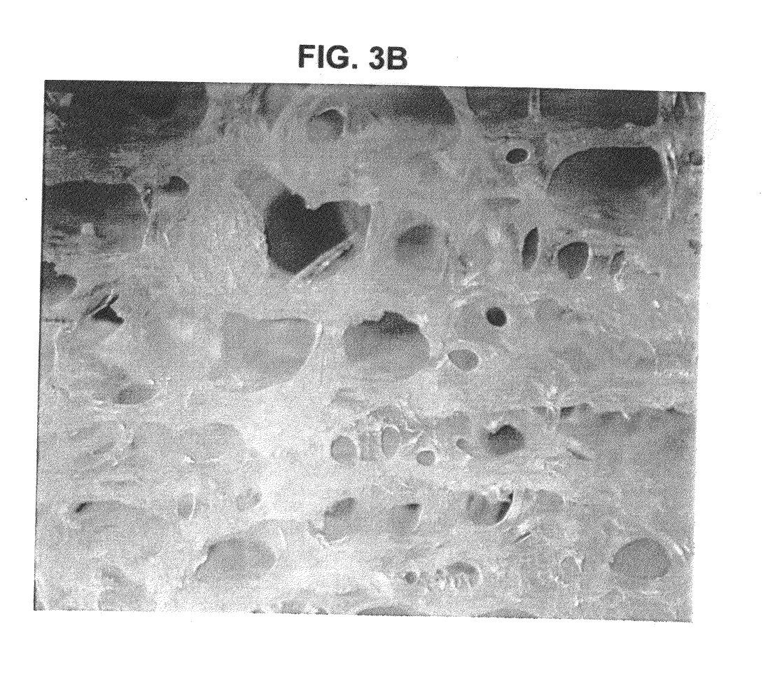

The percentage of porosity and average surface pore diameter of a cancellous construct demineralized cap member according to the present invention can be determined utilizing a microscope/infrared camera and associated computer analysis. A microscope/infrared camera was used to produce the images of FIGS. 3A and 3B, which provide a visual assessment of the porosity of a demineralized cap of a construct according to the present invention. Such images were analyzed using suitable microscopy and image analysis software; for example, Image Pro Plus® (Media Cybernetics, Inc., Bethesda, Md.). The number and diameter of pores and the relative porosity of a demineralized member of a construct can be characterized using techniques known to those skilled in the art.

It is noted that, for allograft constructs, the number and diameter of pores and the relative porosity of the demineralized members will vary from one tissue donor to another, and even within the tissue of one tissue donor, based on the anatomical and/or physical properties of the allograft cancellous bone from which the demineralized member is derived.

### Example 2

### Tissue Extraction and Particularization

A process of cartilage particle extraction may be applied to any of a number of different soft tissue types (for example, meniscus tissue). In an embodiment, cartilage is recovered from deceased human donors, and the tissue is treated with a soft tissue process.

Fresh articular cartilage is removed from a donor using a scalpel, taking care to remove the cartilage so that the full thickness of the cartilage is intact (excluding any bone). Removed cartilage is then packaged in double Kapak® bags for storage until ready to conduct chemical cleaning of the allograft tissue. In one example, the cartilage can be stored in the refrigerator for 24-72 hours or in the freezer (e.g., at a temperature of −70° C.) for longer-term storage.

Chemical cleaning of cartilage tissue is then conducted according to methods known to those skilled in the art. Subsequent to chemical cleaning, the cartilage is lyophilized so as to reduce the water content of the cartilage tissue to within the range of about 0.1% to about 8.0%. Subsequent to the initial lyophilization, the cartilage is freeze-milled, wherein the cartilage is frozen (for example, with liquid nitrogen as a freezing agent) and ground into particles. The cartilage particles are sieved, for example, through a 212 micron sieve.

Next, the lyophilized, freeze-milled cartilage particles are processed into a gel or paste by combining the freeze-milled cartilage particles with PBS.

Exogenous growth factors are optionally added at this stage, and the cartilage particles/exogenous growth factor/PBS mixture is optionally left to equilibrate. Optionally, growth factor may be added to the cartilage particles without or prior to subsequent processing into a gel or paste. The gel or paste may optionally be lyophilized again subsequent to the addition of growth factors.

The cartilage particle gel or paste is then loaded into the demineralized portion of the construct. The amount of cartilage particle gel or paste loaded into the demineralized portion varies, is characterizable by any of a number of methods known to those of ordinary skill in the art, and is dependent at least on such factors as the volume of the demineralized portion of the construct; the average pore size of the demineralized portion; the average porosity of the construct; and the average and median size of the cartilage particles within the cartilage gel or paste.

The cartilage particle gel or paste-loaded construct is then packaged for a second lyophilization step. The cartilage particle gel or paste-loaded construct is lyophilized and may then be provided for surgery, or maintained for later use.

### Example 3

### Extraction of Proteins from Human Cartilage Using Extraction and Subsequent Dialysis

In another example, growth factors may be physically and/or chemically isolated from cartilage particles, and dialyzed using a suitable agent. The growth factors are thereby isolated for subsequent analysis and/or quantification. In an embodiment, 0.3 g of cartilage particles were weighed out for each donor. The cartilage particles were transferred to tubes containing 5 ml of extraction solution (4M guanidine HCl in Tris HCL). The cartilage particles were incubated at 4° C. on an orbital shaker at 60 RPM for 24 hours, followed by dialysis (8 k MWCO membrane dialysis tube) in 0.05M TrisHCL or PBS for 15 hrs. at 4° C. The dialysis solution was then replaced and the dialysis continued for another 8 hrs. at 4° C. The post-dialysis extracts were stored at −70° C. until an Enzyme-linked Immunosorbent Assay (ELISA) was run.

### Example 4

### ELISA of Endogenous Growth Factors in Cartilage Particles

The quantities and concentrations of various endogenous growth factors isolated from cartilage in the Examples 5-10 herein were assessed utilizing ELISA. ELISA may be conducted using any available ELISA protocol, including but not limited to R&D Systems ELISA kits (R&D Systems, Inc., Minneapolis, Minn.) and ProMega's TGF-β Emax™ ImmunoAssay System (ProMega Corporation, Madison, Wis.).

### Example 5

### Quantification of Endogenous Growth Factors Present in Freeze-Milled Cartilage

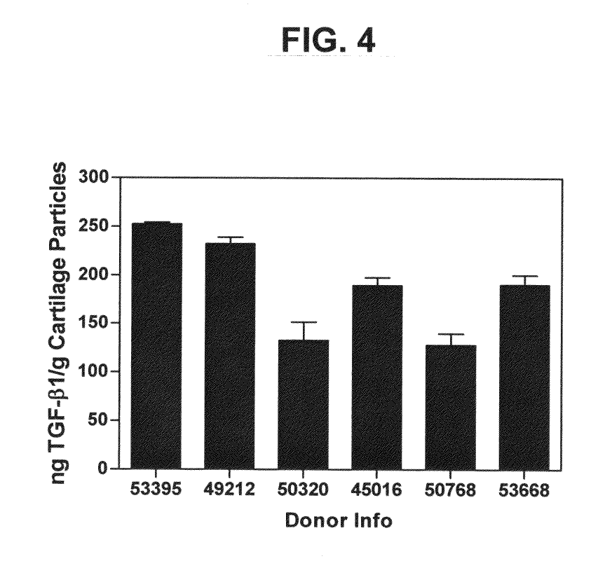

Six respective sets of freeze-milled cartilage particles were prepared from cartilage donated by six tissue donors, according to the method of Example 2 above. 0.3 g. of cartilage particles from each tissue donor were transferred to tubes containing 5 ml of extraction solution (4M guanidine HCl in Tris HCl). The cartilage particles were incubated at 4° C. on an orbital shaker at 60 rpm for 24 hrs, followed by dialysis (8 k MWCO membrane dialysis tube) in 0.05M Tris HCl or PBS for 15 hrs at 4° C. The dialysis solution was then replaced and the dialysis continued for another 8 hrs at 4° C. The post-dialysis extracts were stored at −70° C. until the ELISA was run. Notably, the above protocol can also be utilized in order to determine the total endogenous growth factor concentration present in a device (i.e., construct, scaffold, etc.) of the instant invention. FIG. 4 demonstrates the relative concentration of endogenous total TGF-β1 found in freeze-milled cartilage particles of the present invention derived from the various tissue donors.

### Example 6

### Increased Availability of Endogenous TGF-β1 from Freeze-Milled Cartilage

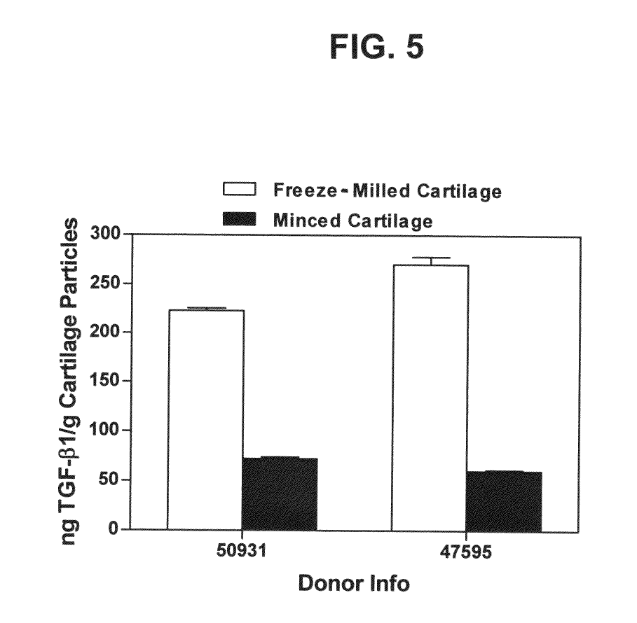

In order to assess the relative amounts of endogenous TGF-β1 accessible via guanidine extraction, the guanidine extraction of endogenous TGF-β1 from minced (e.g., not freeze-milled) cartilage pieces was compared to the guanidine extraction of TGF-β1 from freeze-milled cartilage particles. Increased amounts of endogenous TGF-β1 may be extractable from freeze-milled cartilage particles, as opposed to minced (e.g., not freeze-milled) cartilage pieces. This may be attributable to the increased surface area of the freeze-milled cartilage particles. For example, the fracture planes; three-dimensional shape of the particles; and resulting increased surface area may enhance the release of the cartilage growth factors or other substances from the particles, or enhance the accessibility of growth factors to surrounding cells. This may influence bioavailability of endogenous growth factors and activation of latent endogenous growth factors. Furthermore, the avoidance of elevated temperatures during processing may facilitate the production of particles having high chondrogenic activity by facilitating substantial preservation of extracellular matrix components. For example, preservation of the required tertiary or quaternary folding structures of endogenous growth factors or other proteins in tissue subjected to freeze-milling may occur. FIG. 5 provides an indication of the relative amounts of growth factor that were isolated from minced cartilage and from freeze-milled cartilage particles of the present invention, respectively.

### Example 7

### Quantification of Total Endogenous FGF-2 Present in Freeze-Milled Cartilage Particles

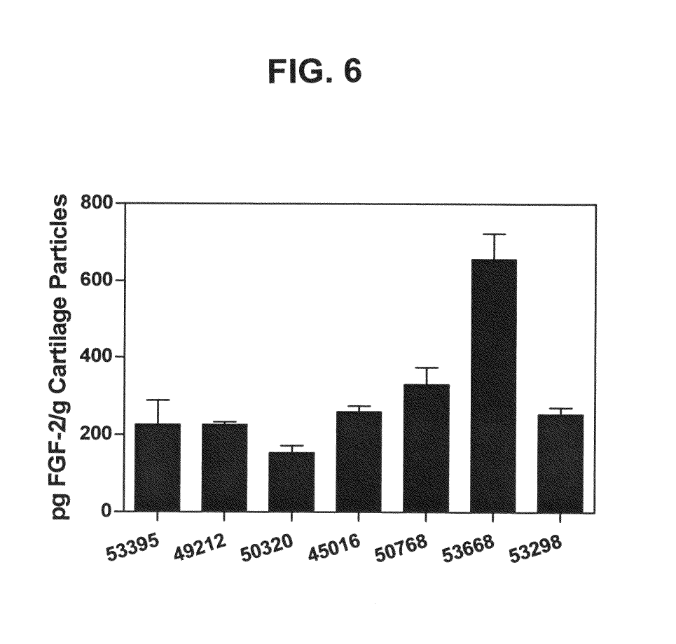

FIG. 6 demonstrates the relative concentration of endogenous FGF-2 found in freeze-milled cartilage particles of the present invention that were prepared in accordance with Example 2 of the present invention and derived from various tissue donors.

### Example 8

### Quantification of Total Endogenous BMP-2 Present in Freeze-Milled Cartilage Particles

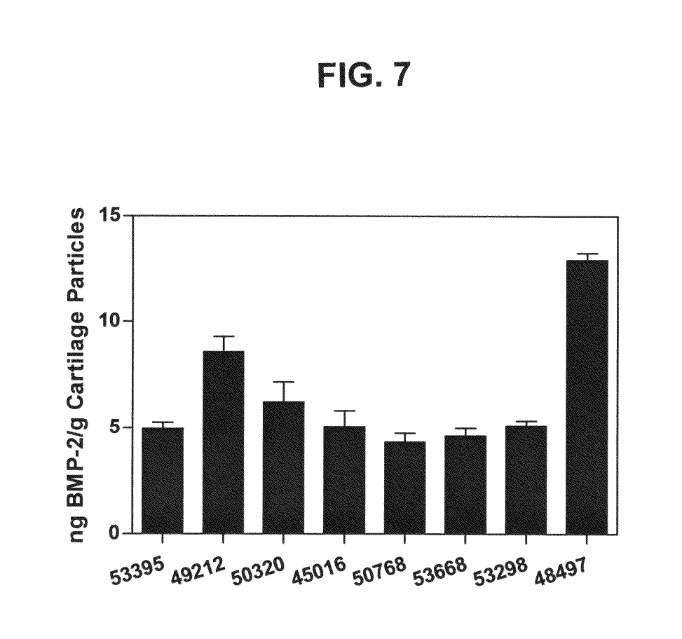

FIG. 7 demonstrates the relative concentration of endogenous BMP-2 found in freeze-milled cartilage particles of the present invention prepared in accordance with Example 2 of the present invention and derived from various tissue donors.

### Example 9

### Quantification of Total Endogenous GDF-5/BMP-14 Present in Freeze-Milled Cartilage Particles

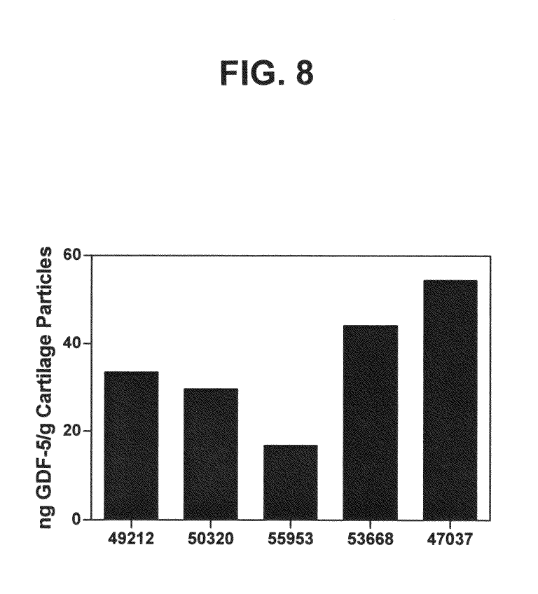

FIG. 8 demonstrates the relative concentration of endogenous GDF-5/BMP-14 found in freeze-milled cartilage particles of the present invention prepared in accordance with Example 2 of the present invention and derived from various tissue donors.

### Example 10

### Quantification of Total Endogenous IGF-1 Present in Freeze-Milled Cartilage Particles

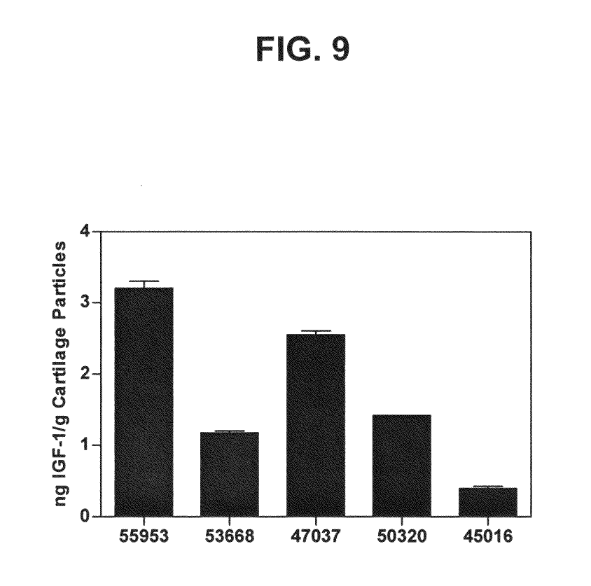

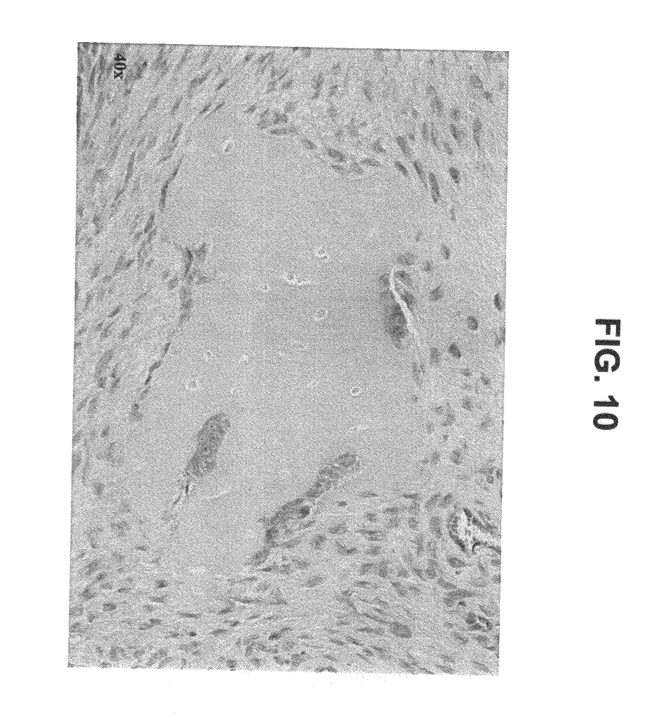

FIG. 9 demonstrates the relative concentration of endogenous IGF-1 found in freeze-milled cartilage particles of the present invention prepared in accordance with Example 2 of the present invention and derived from various tissue donors.

As shown in the included tables and figures, freeze-milled cartilage particles and minced cartilage retain a concentration of endogenous TGF-β1. Such concentration of TGF-β1 is more bioavailable in the freeze-milled particles described herein. Freeze-milled cartilage particles as described herein also retain a concentration of endogenous BMP-2; BMP-14/GDF-5; IGF-1; and FGF-2.

### Example 11

### Relative Efficacy of Various Cartilage Paste and Clinical Standard Methods in Particular Cartilage Reconstruction In Vivo (Microfracture)

An animal study was conducted using critical-sized defects in Spanish goats to determine the cartilage-healing potential of the three cartilage paste preparations listed in TABLE 1 below. Chondral defects were created on the medial femoral condyle (6 mm diameter) and trochlear sulcus (5 mm diameter) followed by microfracture and implantation of the cartilage paste.

TABLE 1

| | | | # of | |
|---|---|---|---|---|
| Implant | Procedure | Splint Time | Goats | Study Time |
| Particles | Microfracture | 3, 7, or | 3 | 6 weeks |
| in PBS | | 14 days | | |
| Particles in Hy | Microfracture | 3, 7, or | 3 | 6 weeks |
| | | 14 days | | |
| Particles in Hy + | Microfracture | 3, 7, or | 3 | 6 weeks |
| Insulin | | 14 days | | |

All of the animals exhibited some circumferential healing in one or more of the defects. Healing ranged from 0 to 75% in the medial femoral condyle lesion areas being filled with repair tissue, and from 0 to 90% in the trochlear sulcus lesion areas being filled with repair tissue.

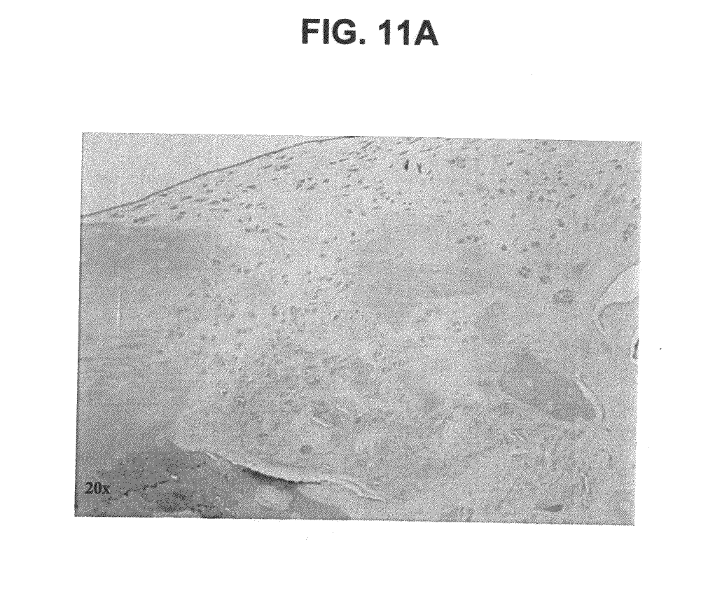

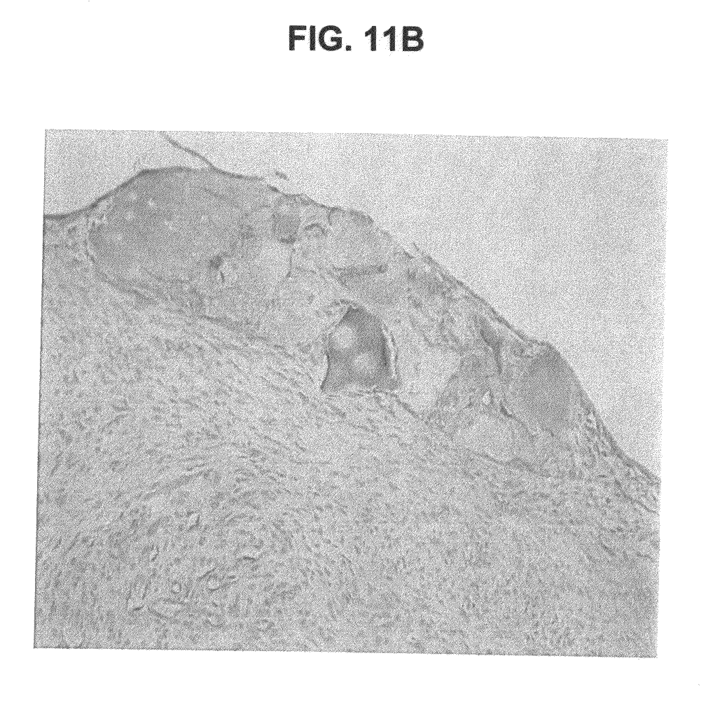

Histologically, it was observed that, where the cartilage paste was retained in the defect, the repair tissue was positive for glycosaminoglycans (GAG) and collagen II, which indicated the presence of articular cartilage. This occurred most frequently around the edges of the defect, where the cartilage paste was more likely to stay in place. FIG. 10 demonstrates infiltration of lacunae by chondrocytes. FIGS. 11A and 11B demonstrate residual and new collagen type II, respectively. Features shown in FIGS. 10, 11A and 11B were visualized via immunohistochemistry.

### Example 12

### Comparative In Vivo Study of Articular Cartilage Regeneration of Induced Osteochondral Defects

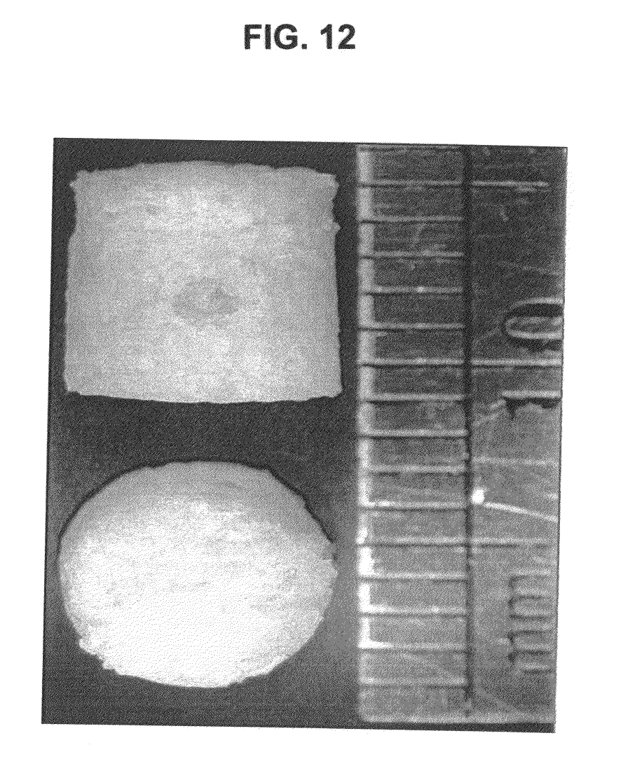

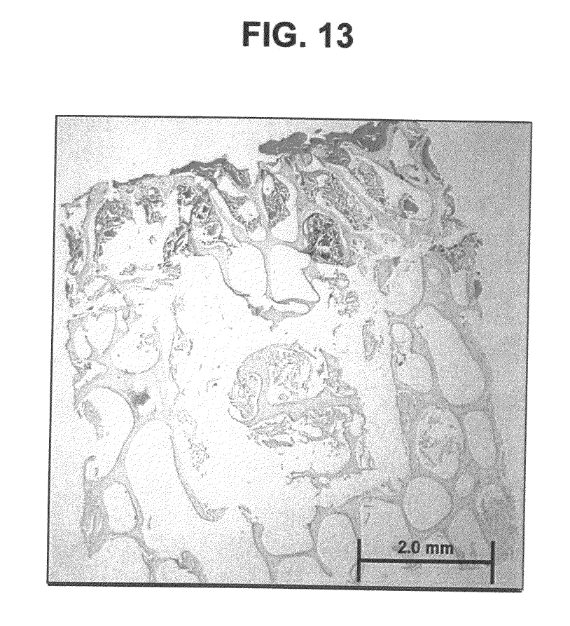

An in vivo animal study was conducted on critical sized defects in Spanish goats, utilizing constructs such as disclosed herein combined with various cartilage particle preparations. FIG. 12 is a photographic depiction of a construct such as disclosed herein and utilized in the study. FIG. 13 depicts homogenous distribution of infused cartilage particles in a construct, as determined by Safranin-O (proteoglycan) staining.

TABLE 2 below details the content of each implant used in the study, each of which was assayed in duplicate (12 and 24 weeks duration in vivo implantation). “MFX” refers to the microfracture procedure performed in the defect that was used as a control (i.e., without the implantation of constructs or cartilage particles). “ACS” refers to “allograft cartilage scaffold”, incorporating embodiments of both the cartilage particles and the constructs of the instant application.

TABLE 2

| | | Splint Time | # of | Survival | |
|---|---|---|---|---|---|
| Group # | Implant | (days) | Defects | (weeks) | # Animals |
| 1 | Empty Defect | 7 | 2 | 12, 24 | 2 |
| 2 | MFX Control | 7 | 2 | 12, 24 | 2 |
| 3 | ACS No | 7 | 2 | 12, 24 | 5 |
| | Particles | | | | |
| 4 | ACS | 7 | 2 | 12, 24 | 5 |

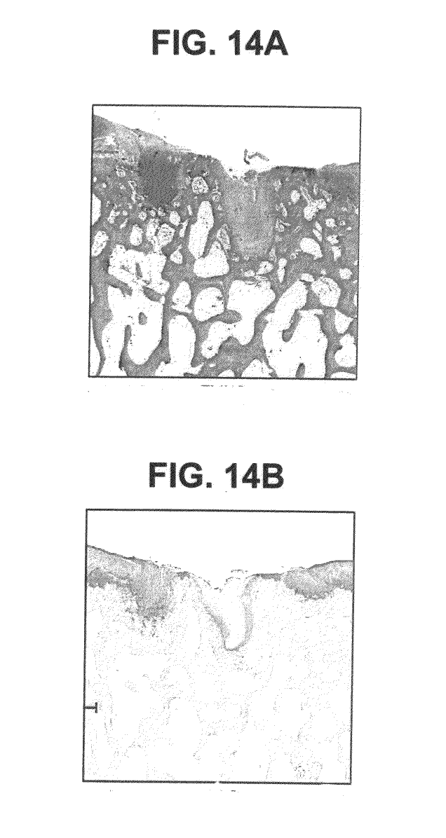

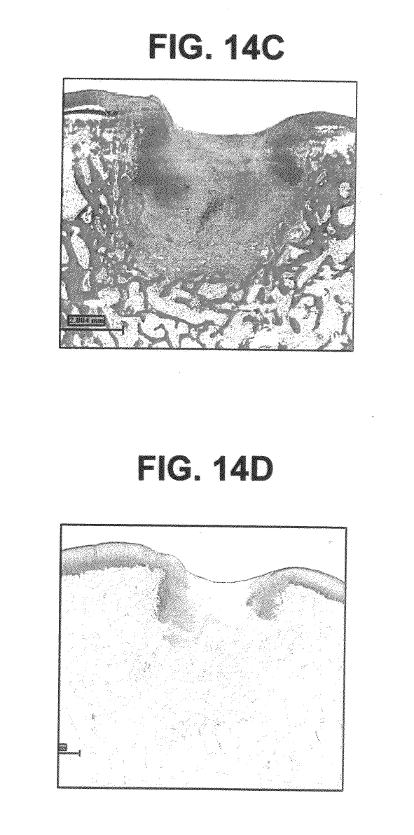

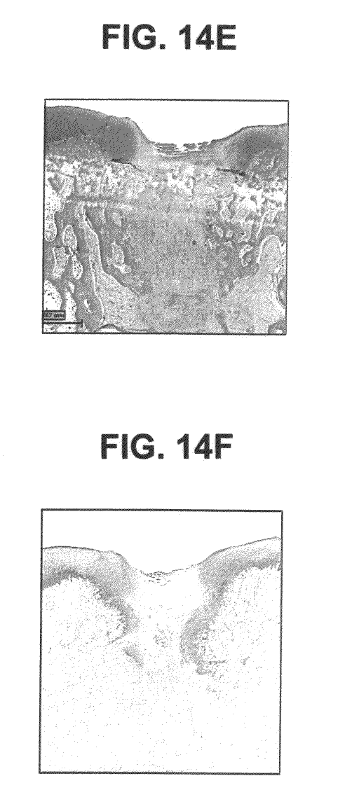

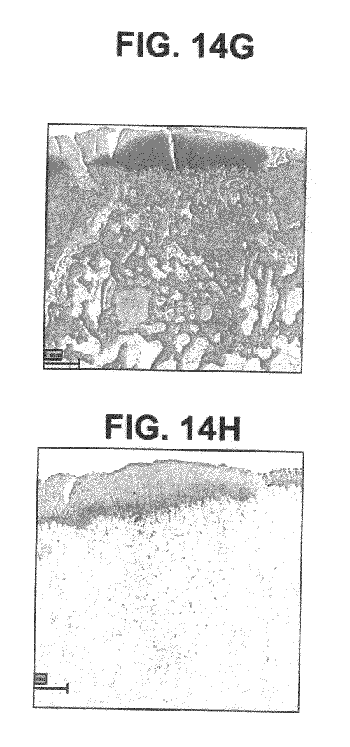

FIGS. 14A-14H demonstrate improved and selective chondrogenesis when constructs and freeze-milled cartilage particles of the instant invention are used in conjunction with each other. FIGS. 14A, 14C, 14E and 14G are stained with Safranin-O for proteoglycan assessment. FIGS. 14B, 14D, 14F and 14H show tissues stained with anti-collagen II antibodies. FIGS. 14A and 14B depict microfracture (Group 2); FIGS. 14C and 14D depict an empty defect (Group 1); FIGS. 14E and 14F depict a construct without freeze-milled cartilage particles (Group 3); and FIGS. 14G and 14H depict a construct with infused cartilage particles. (Group 4).

### Example 13

### FGF2v1 Release Profile from Cartilage Paste Reconstructed with PBS

A study was conducted to follow the release of FGF2v1 adsorbed to a cartilage paste of freeze-milled cartilage particles (as prepared as in Example 2 above) in PBS. The basic formula for preparing the paste was to add 780 μl of PBS for each 220 mg of cartilage particles. The paste was centrifuged and excess liquid was removed, then the particles were resuspended in 0.5 mL of PBS containing FGF2v1 and shaken at 4° C. to adsorb FGF2v1 to the cartilage particles. Pastes were prepared at FGF2v1 concentrations of 0, 0.5, 5, 50 and 500 μg FGF2v1/ml PBS.

To measure the FGF2v1 that had been adsorbed to the cartilage particles (without the non-adsorbed FGF2v1 remaining in the aqueous phase or trapped in the void volume of the paste), the paste was thoroughly washed 5 times in PBS. The washing solution was collected and analyzed for FGF2v1.

Two 20 mg samples were prepared from each paste. The samples were each re-suspended in 1 ml PBS and shaken at 4° C. for a number of days. The samples were then centrifuged and the liquid phase was collected for analysis of FGF2v1. The samples were then re-suspended in 1 ml PBS and the shaking and liquid collection steps were repeated. At the end of the test, the cartilage particles were digested by collagenase degradation and the liquid phase was analyzed for FGF2v1.

FGF2v1 in the liquid phase was measured by two methods: (i) ELISA was used to measure total amounts of FGF2v1 protein; and (ii) a functional cell-based assay (FDCP) was used to measure active amounts of FGF2v1.

The ELISA procedure was carried out using a commercial ELISA development kit for human FGF-2 (R&D Systems, Inc., Minneapolis, Minn.; Catalog Number: DY233). Solutions of FGF2v1 were used as standards.

The FDCP assay was performed using cells that express FGFR-R1 and require activation by FGF to proliferate. Quantification of cell proliferation was based on the ability of metabolically-active cells to reduce tetrazolium salt XTT substrate (Biological Industries Israel, Kibbutz Beit Heimek, Israel; Catalog Number 20-300-1000) to orange-colored compounds of formazan. The dye formed is water-soluble and the dye intensity can be read at a given wavelength with a spectrophotometer. The intensity of the dye is proportional to the number of metabolically-active cells. The cell line used in the assay and the assay procedure were developed by ProChon Biotech Ltd., Rehovot, Israel.

In the FDCP assay, 50 μl of growth media containing 2×10⁴ FDCP cells expressing FGFR-R1 were cultivated with 50 μl of PBS (containing FGF2v1) collected from the release experiment described above. Before the FGF2v1 was applied, it was serially diluted in growth media in 96-well flat-bottom plates. The plates were incubated in a 5% CO₂ incubator at 37° C. for 48 hours. 50 μl of XTT were then added to each well and the plate was again incubated for 6 hours. The plate was then shaken gently to evenly distribute the dye within each well and the absorbance of the samples was measured at a wavelength of 450-500 nm. Quantification of the active FGF2v1 was made by comparison to a standard calibration curve.

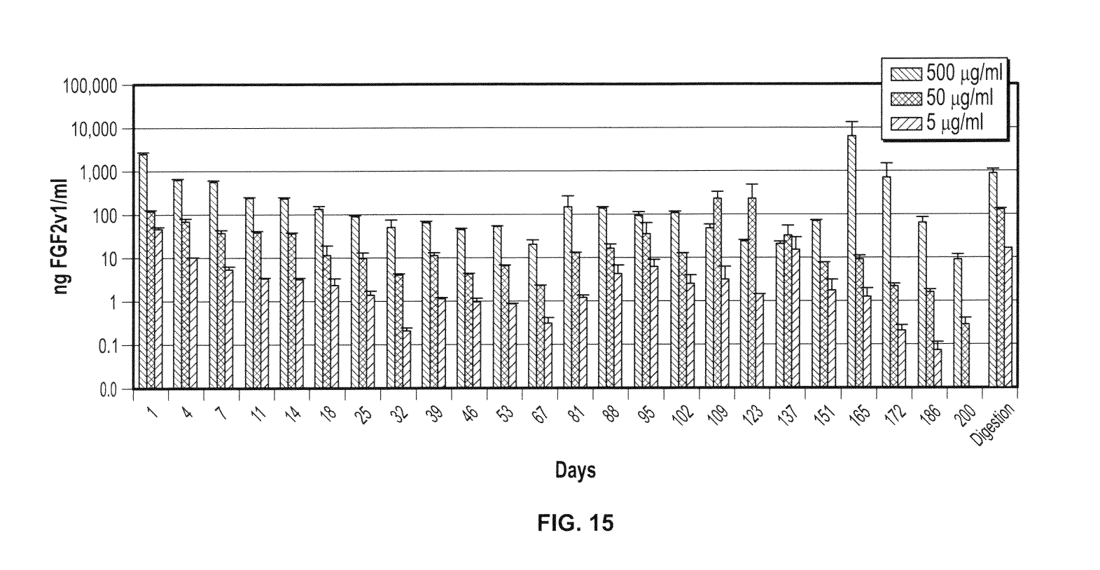

FIG. 15 presents the release of total FGF2v1 from the cartilage paste over time as measured by ELISA. Data represent the averaged assayed FGF2v1 concentrations from the 20 mg samples exposed to 5, 50 and 500 μg FGF2v1/ml of PBS. The assay results for the 20 mg samples exposed to 0.5 μg FGF2v1/ml of PBS were below quantification levels and are, therefore, not presented. The control samples (i.e., 20 mg samples exposed to PBS without FGF2v1) did not show any FGF2v1 activity, as expected. Total amounts of FGF2v1 equivalent to 92-100% of the applied FGF2v1 were measured in the liquids collected during the washes, the release experiments, and the digestion of the cartilage particles for cartilage paste samples exposed to initial FGF2v1 concentrations of 5, 50 and 500 μg FGF2v1/ml of PBS.

Examination of FIG. 15 shows that the FGF2v1 was slowly released from the cartilage paste for all samples, with the release continuing for at least 180 days. For the samples exposed to 50 or 500 μg FGF2v1/ml of PBS, the release rate was substantially the same at about day 165 of the study as during the early weeks of the study.

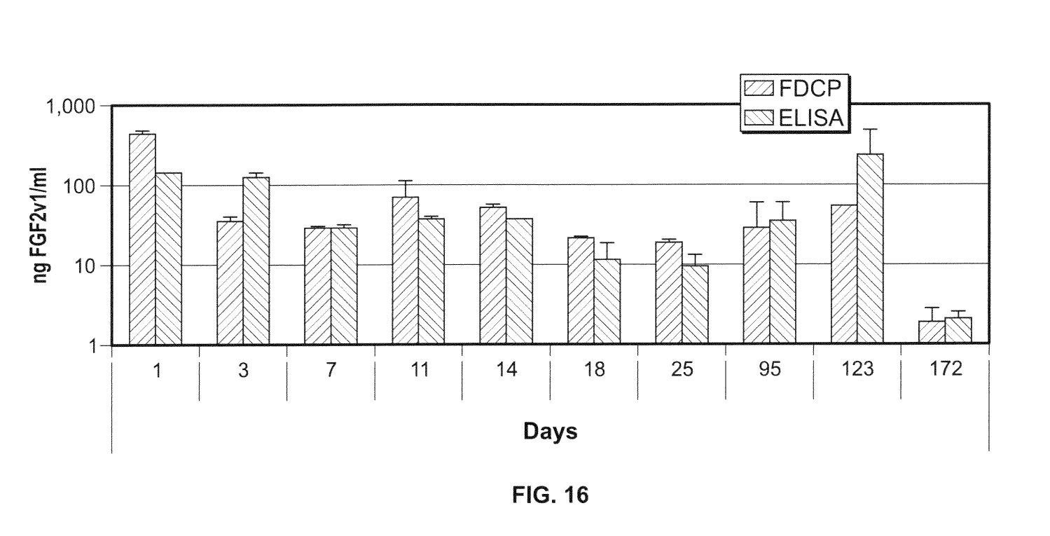

FIG. 16 compares the results of the ELISA and FDCP assays of FGF2v1 released from the 20 mg samples of cartilage paste exposed to an initial concentration of 50 μg FGF2v1/ml of PBS. As discussed above, the ELISA measures the amount of total FGF2v1 released from the cartilage paste, and the FDCP assay measures the amount of active FGF2v1 released from the cartilage paste. The results of the two assays correlate well, indicating that the majority of the released FGF2v1 is active.

### Example 14

### Proliferation of Chondrocytes in Cancellous Bone Constructs

Chondrocytes will proliferate within cancellous bone constructs of the types disclosed herein and in the aforementioned U.S. Patent Application Publication No. 2009/0319045. Human articular chondrocytes were isolated from fresh human cartilage by enzymatic digestion. The isolated chondrocytes were expanded in monolayer and then seeded on cancellous bone strips further comprising fibrin. After 5 days of incubation in chondrocyte expansion medium, the cancellous bone strips were examined by histology using Hematoxylin and Eosin stain (H&E). Histological sections showed high chondrogenic proliferation of human articular chondrocytes seeded on the cancellous bone.

### Other Examples

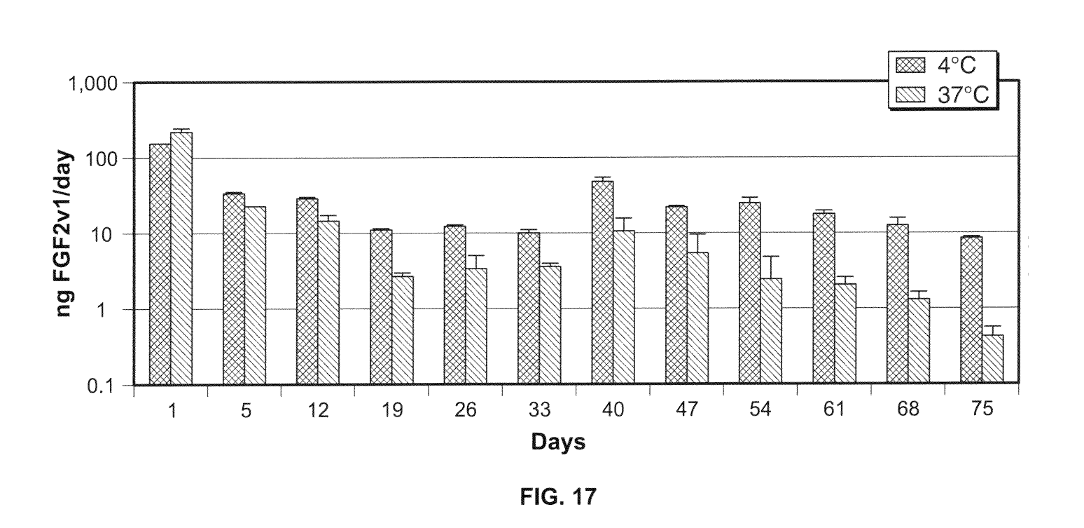

Other examples of release and stability of FGF in cartilage pastes and cell seeding of cancellous bone are briefly discussed herein with reference to FIGS. 17-34.

FIG. 17 presents the release of total FGF2v1 from a paste of lyophilized freeze-milled cartilage paste over time, as measured at 4° C. and 37° C. Data represent the daily released amounts of FGF2v1 at both temperatures. Release of FGF2v1 was observed at both temperatures for at least 75 days.

Examination of FIG. 17 shows that the amounts of FGF2v1 released from the cartilage particles for the first 12 days was approximately the same at both temperatures. Beginning at about day 19, the amount released at 37° C. was significantly less than the amount released at 4° C. Even greater differences in the relative amounts of FGF2v1 released from the cartilage particles were observed after 40 days.

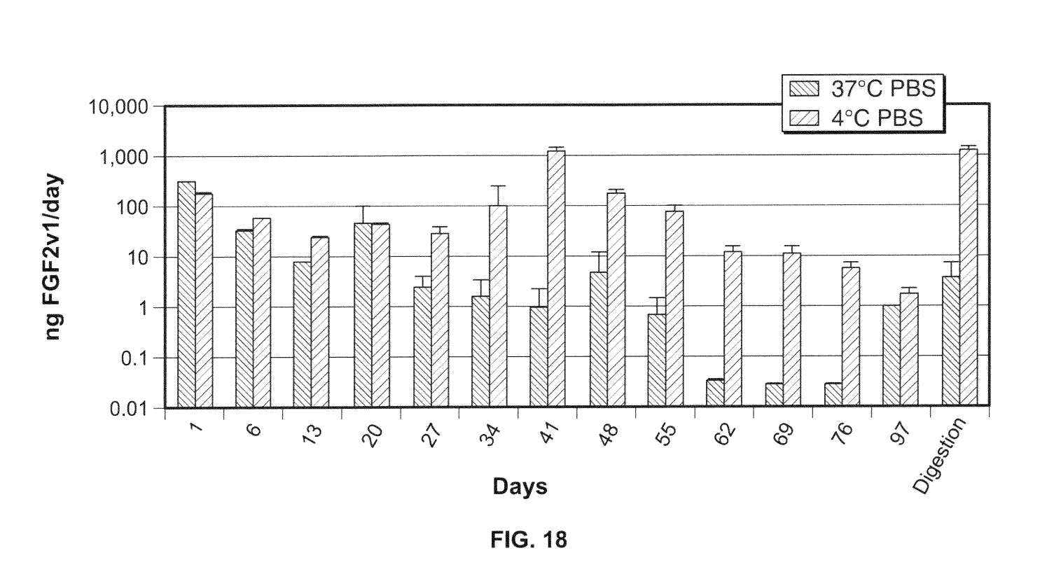

FIG. 18 presents the release of total FGF2v1 from lyophilized, freeze-dried cartilage paste with PBS over time, as measured at 4° C. and 37° C. Data represent the daily released amounts of FGF2v1 at both temperatures. Release of FGF2v1 was observed at both temperatures for at least 97 days and at digestion of the cartilage particles for cartilage paste samples.

Examination of FIG. 18 shows that the amounts of FGF2v1 released from the cartilage particles for the first 20 days was approximately the same at both temperatures. Beginning at about day 27, the amount of FGF2v1 released at 37° C. was significantly less than the amount released at 4° C. Even greater differences in the relative amounts of FGF2v1 released from the cartilage particles were observed from day 62 to day 76, but at day 97, the amounts of FGF2v1 released were about the same at both temperatures. Substantially less FGF2v1 was recovered by digestion from the particles held at 37° C. relative to those held at 4° C.

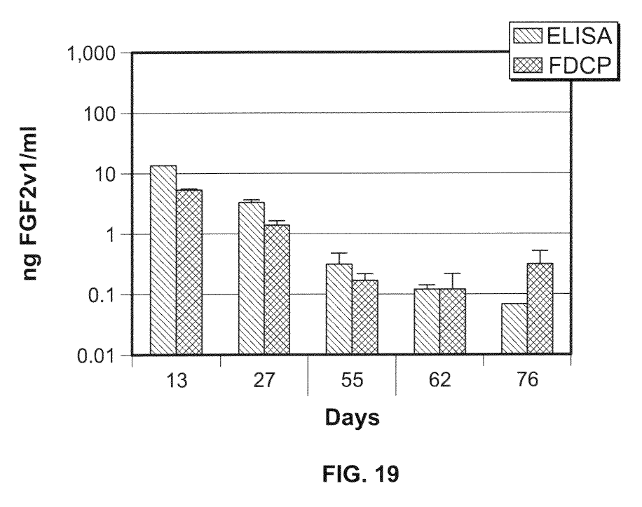

FIG. 19 compares the results of the ELISA and FDCP assays of FGF2v1 released from cartilage paste at 37° C. As discussed above, the ELISA measures the amount of total FGF2v1 released from the cartilage paste, and the FDCP assay measures the amount of active FGF2v1 released from the cartilage paste. The results of the two assays on different days correlate well, indicating that the majority of the released FGF2v1 is active.

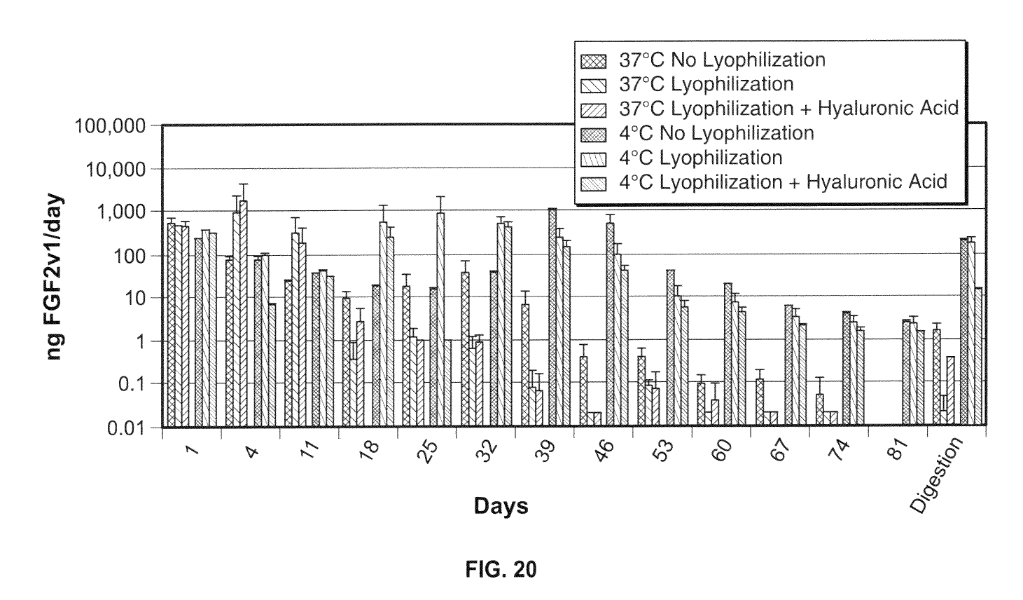

FIG. 20 presents the release of total FGF2v1 from three groups of reconstituted freeze-dried cartilage paste over time, as measured at 4° C. and 37° C. The three groups included cartilage paste containing non-lyophilized cartilage particles; cartilage paste containing lyophilized cartilage particles; and cartilage paste containing lyophilized cartilage particles and hyaluronic acid. The cartilage paste also included PBS and 1% bovine serum albumin (BSA) in all of the groups. Data represent the daily released amounts of FGF2v1 from the three groups, at both temperatures. Release of FGF2v1 was observed at both temperatures for at least 81 days and at digestion of the cartilage particles for cartilage paste samples. Over most of the test period, the amount of FGF2v1 measured for the samples held at 37° C. were substantially less than the amounts measured for the samples held at 4° C. Substantially less FGF2v1 was recovered by digestion from the samples held at 37° C. than from the samples held at 4 C.

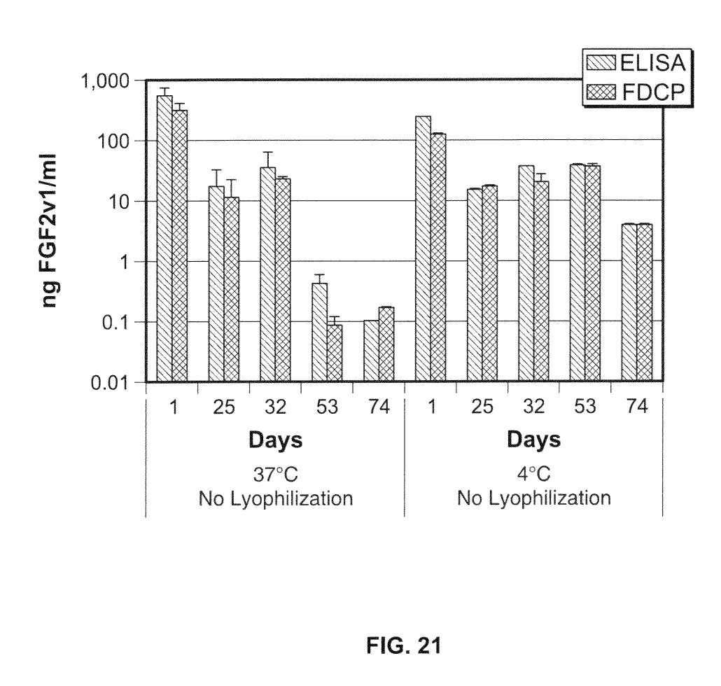

FIG. 21 compares the results of the ELISA and FDCP assays of FGF2v1 released from cartilage paste at 37° C. to results of the ELISA and FDCP assays of FGF2v1 released from cartilage paste at 4° C. As discussed above, the ELISA measures the amount of total FGF2v1 released from the cartilage paste, and the FDCP assay measures the amount of active FGF2v1 released from the cartilage paste. The results of the two assays on each day correlate well within the set of data obtained at each temperature, indicating that the majority of the released FGF2v1 is active.

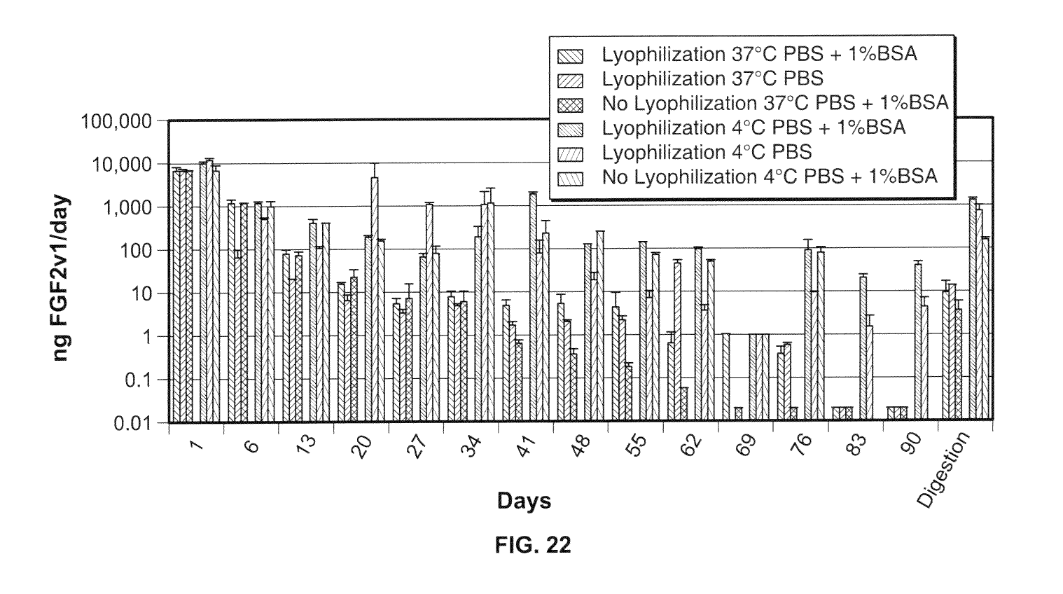

FIG. 22 presents the release of total FGF2v1 from three groups of freeze-milled cartilage paste over time, as measured at 4° C. and 37° C. The three groups included cartilage paste containing lyophilized cartilage particles, PBS and 1% BSA; cartilage paste containing lyophilized cartilage particles and PBS; and cartilage paste containing non-lyophilized cartilage particles, PBS and 1% BSA. Data represent the daily released amounts of FGF2v1 from the three groups, at both temperatures. Release of FGF2v1 was observed at both temperatures for at least 90 days and at digestion of the cartilage particles for cartilage paste samples. Over most of the test period, substantially less released FGF2v1 was measured for the samples held at 37° C. than for the samples held at 4° C. Substantially less FGF2v1 was recovered by digestion from the samples that were held at 37° C. than from the samples held at 4 C. However, variations in the use of lyophilization or the presence of BSA did not significantly affect the release rates of FGF2v1 at either temperature.

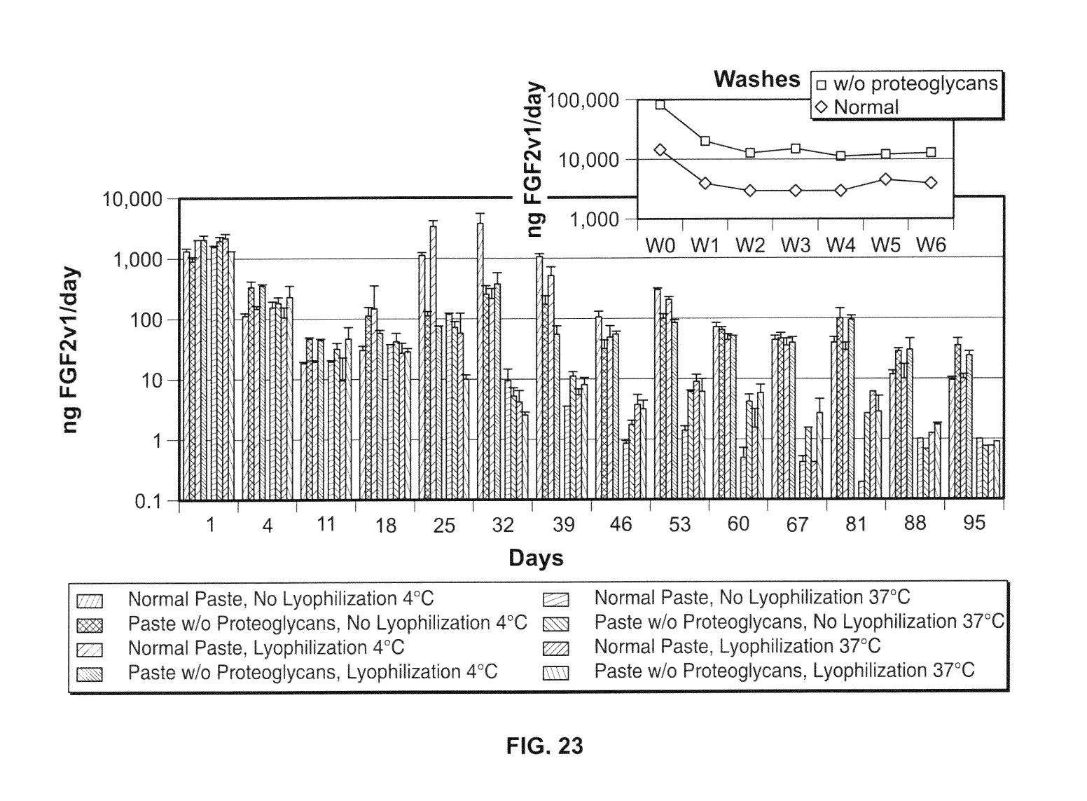

FIG. 23 compares the release of total FGF2v1 from standard, or normal cartilage powder paste and from proteoglycan-free cartilage powder paste. Four groups of cartilage paste were tested over time, as measured at 4° C. and 37° C. The four groups included normal cartilage paste containing lyophilized cartilage particles; normal cartilage paste containing non-lyophilized cartilage particles; proteoglycan-free cartilage paste containing lyophilized cartilage particles; and proteoglycan-free cartilage paste containing non-lyophilized cartilage particles. Data represent the daily released amounts of FGF2v1 from the four groups, at both temperatures. Release of FGF2v1 was observed at both temperatures for at least 95 days. From about day 25 through day 95, substantially less released FGF2v1 was measured for the samples held at 37° C. than from the samples held at 4° C. No consistent differences in the amounts of FGF2v1 released were observed within groups of samples having variations in lyophilization or presence of proteoglycans.

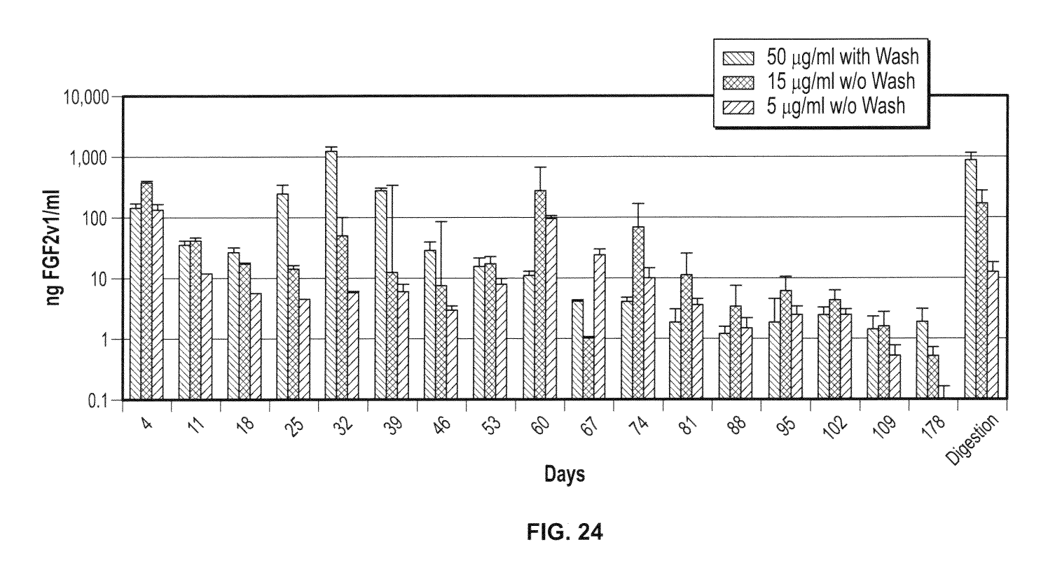

FIG. 24 presents a comparison of the release of FGF2v1 from FGF2v1/PBS cartilage pastes having the following initial concentrations and washings: 50 μg FGF2v1/ml of PBS, with particle washing; 15 μg FGF2v1/ml of PBS, without particle washing; and 5 μg FGF2v1/ml of PBS, without particle washing. Data represent the averaged assayed FGF2v1 concentrations exposed to the respective concentrations of 50, 15, and 5 μg FGF2v1/ml of PBS. Release of FGF2v1 was observed for at least 178 days and at digestion of the cartilage particles for cartilage paste samples.

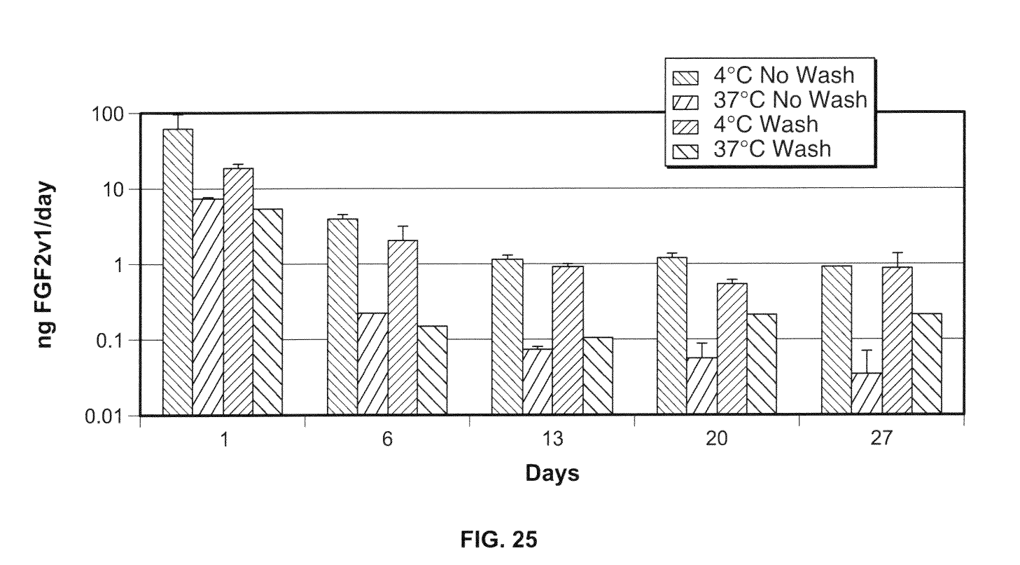

FIG. 25 presents the release of total FGF2v1 from two groups of goat cartilage particles absorbed with FGF2v1 over time, as measured at 4° C. and 37° C. The two groups included washed cartilage particles and unwashed cartilage particles. Data represent the daily released amounts of FGF2v1 from the two groups, at both temperatures. Release of FGF2v1 was observed at both temperatures for at least 27 days.

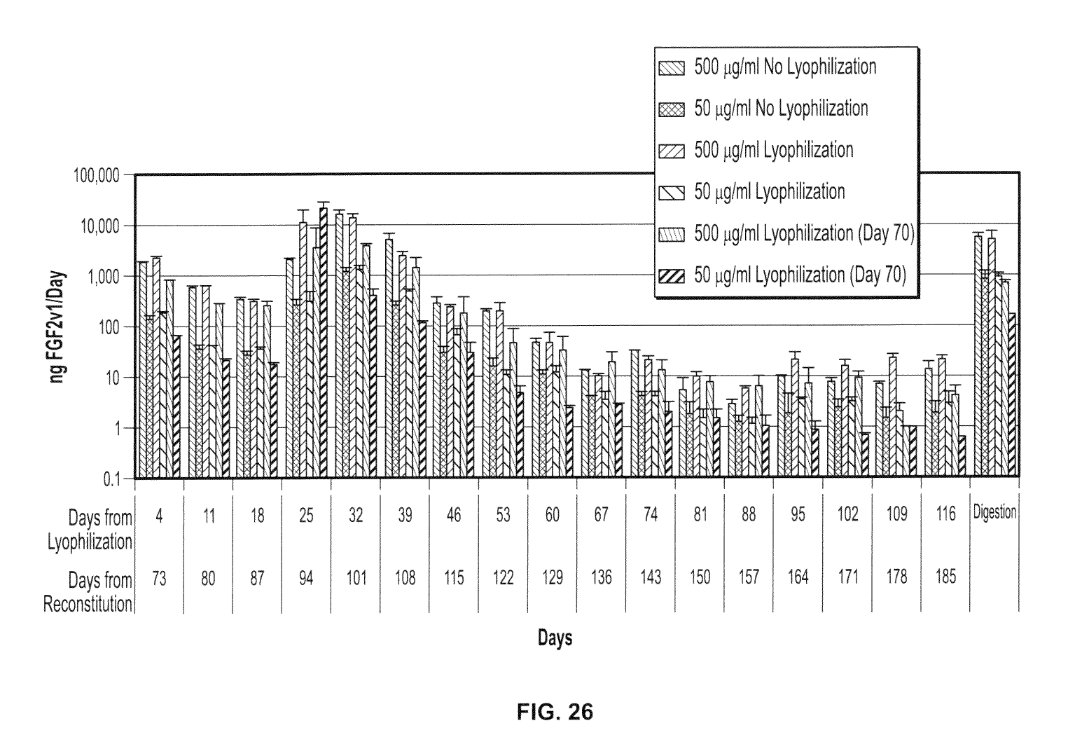

FIG. 26 presents the release of total FGF2v1 from six groups of reconstituted freeze-dried cartilage paste over time. The six groups included cartilage paste containing non-lyophilized cartilage particles and having a concentration of 500 mg FGF2v1/ml of carrier; cartilage paste containing non-lyophilized cartilage particles and having a concentration of 50 μg FGF2v1/ml of carrier; cartilage paste containing lyophilized cartilage particles and having a concentration of 500 μg FGF2v1/ml of carrier; cartilage paste containing lyophilized cartilage particles and having a concentration of 50 μg FGF2v1/ml of carrier; cartilage paste containing cartilage particles that were lyophilized at Day 70 of the study and having a concentration of 500 μg FGF2v1/ml of carrier; and cartilage paste containing cartilage particles that were lyophilized at Day 70 of the study and having a concentration of 50 μg FGF2v1/ml of carrier. Data represent the daily released amounts of FGF2v1 from the six groups, for both days from lyophilization and days from reconstitution. Release of FGF2v1 was observed for at least 116 days from lyophilization, at least 185 days from reconstitution, and at digestion of the cartilage particles for cartilage paste samples.

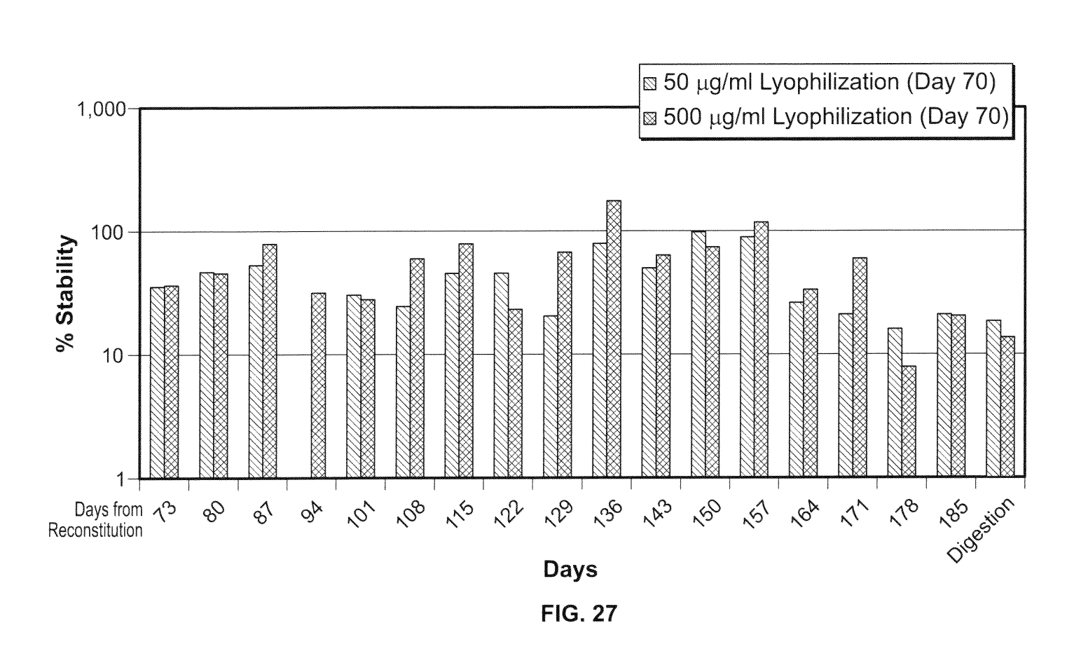

FIG. 27 compares the stability of FGF2v1 in a lyophilized cartilage powder for two groups of cartilage paste over time. The two groups included cartilage paste containing cartilage particles that were lyophilized at Day 70 of the study and having a concentration of 500 μg FGF2v1/ml of carrier; and cartilage paste containing cartilage particles that were lyophilized at Day 70 of the study and having a concentration of 50 μg FGF2v1/ml of carrier. FGF2v1 was observed to be stable for at least 185 days from reconstitution.

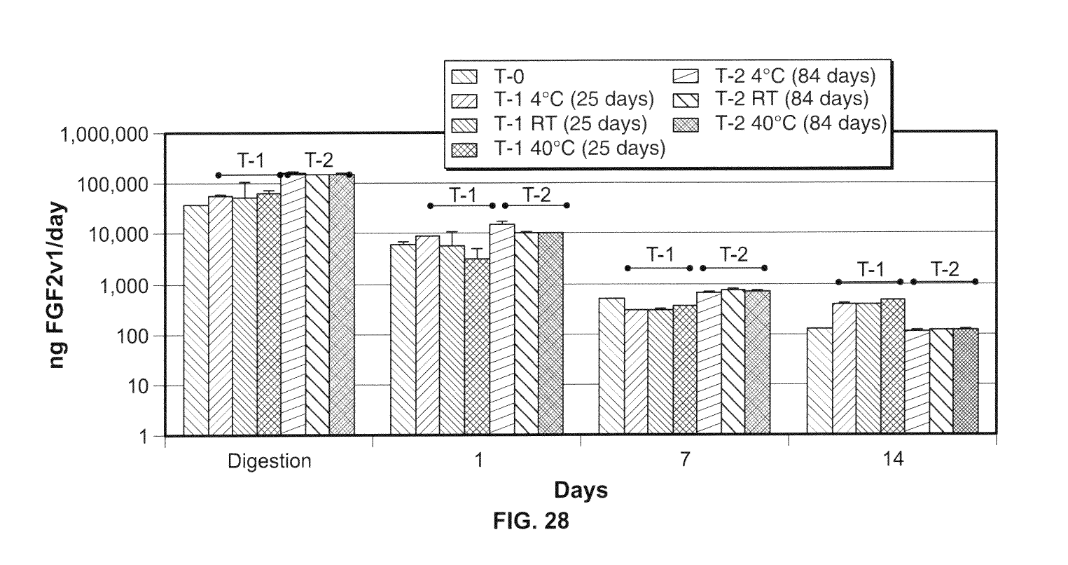

FIG. 28 compares stability of FGF2v1 in six groups of cartilage paste containing a lyophilized cartilage powder/hyaluronic acid formulation over time, wherein the groups were stored for different time periods and at different temperatures. The six groups included cartilage paste that was stored at 4° C. for 25 days; cartilage paste that was stored at room temperature for 25 days; cartilage paste that was stored at 40° C. for 25 days; cartilage paste that was stored at 4° C. for 84 days; cartilage paste that was stored at room temperature for 84 days; and cartilage paste that was stored at 40° C. for 84 days. Release of FGF2v1 was observed for at least 14 days and at digestion of the cartilage particles for cartilage paste samples. A control sample of cartilage paste was used that had not been stored for any length of time. The amounts of FGF2v1 released by the samples did not vary significantly with storage time or storage temperature, and were similar to the amounts released from the control sample.

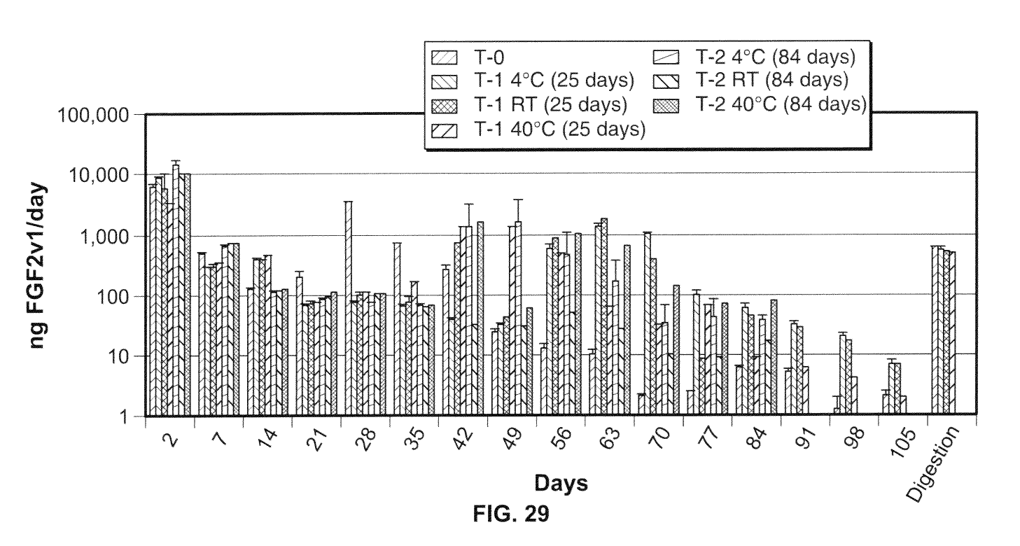

FIG. 29 presents the results of a study similar to that illustrated in FIG. 28, but conducted over a longer period of time. As in FIG. 28, the release of FGF2v1 from six groups of cartilage paste containing a lyophilized cartilage powder/hyaluronic acid formulation over time, wherein the groups were stored for different time periods and at different temperatures. The six groups included cartilage paste that was stored at 4° C. for 25 days; cartilage paste that was stored at room temperature for 25 days; cartilage paste that was stored at 40° C. for 25 days; cartilage paste that was stored at 4° C. for 84 days; cartilage paste that was stored at room temperature for 84 days; cartilage paste that was stored at 40° C. for 84 days. Release of FGF2v1 was observed for at least 105 days and at digestion of the cartilage particles for cartilage paste samples. A control sample of cartilage paste was used that had not been stored for any length of time. The results for each time period did not vary significantly between the three storage temperatures until about Day 35. After Day 35, greater differences in the relative amounts of released FGF2v1 were observed for the different storage temperatures within each time period.

Based on the studies and data discussed above and illustrated in FIGS. 15-29, the following conclusions may be drawn regarding the combination of cartilage paste and FGF2v1:

(a) FGF2v1 is adsorbed to the cartilage paste that retains approximately 70-80% of the FGF2v1 presented in the solution;

(b) Cartilage paste that is maintained at 4° C. releases measurable amounts of FGF2v1 over an extended period of time;

(c) There is a dose effect that correlates the release to the amount of FGF2v1 introduced into the paste;

(d) FGF2v1 released from the cartilage paste is active, and correlates to the total FGF2v1 levels measured by ELISA;

(e) A stabilizer is not needed to retain FGF2v1 in the cartilage paste;

(f) Similar FGF2v1 release profiles at 4° C. and 37° C. were observed during the first two to three weeks of the respective studies. Decreased levels of released FGF2v1 at 37° C. were observed at later times;

(g) The lyophilized paste with FGF2v1 is stable over a period of up to two months.

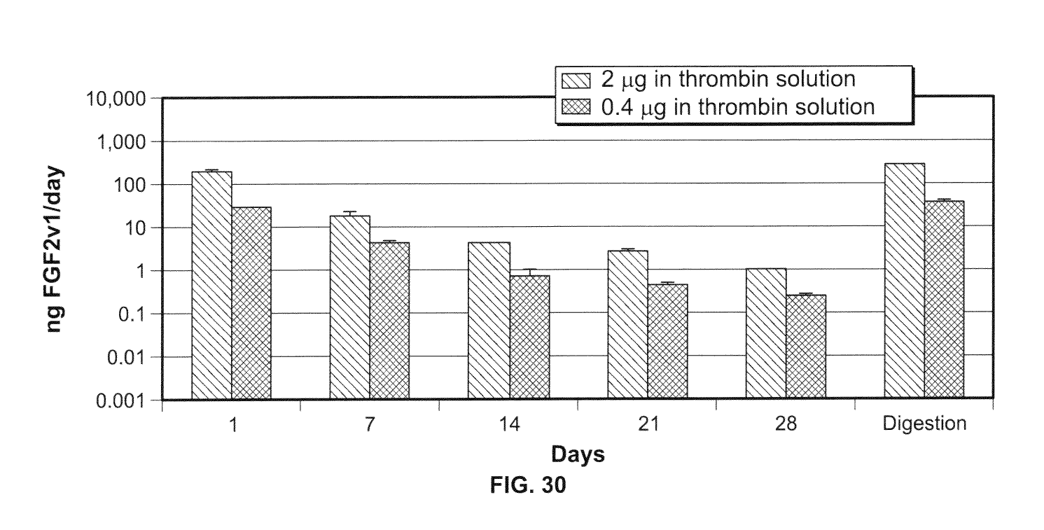

FIG. 30 presents the release of FGF2v1 from two groups of a combination of human cartilage powder and a fibrinogen scaffold over time. Human cartilage powder was prepared as in Example 2. In the a first sample, cartilage powder was then loaded with 2 μg of FGF2v1 per ml of cartilage powder. In a second sample, cartilage powder was loaded with 0.4 μg of FGF2v1 per ml of cartilage powder. Both groups were then mixed with a thrombin solution and fibrin to form a fibrin matrix from which FGF2v1 release was measured. The data presented in FIG. 30 represent the daily released amounts of FGF2v1 from the two groups. Release of FGF2v1 was observed for at least 28 days.

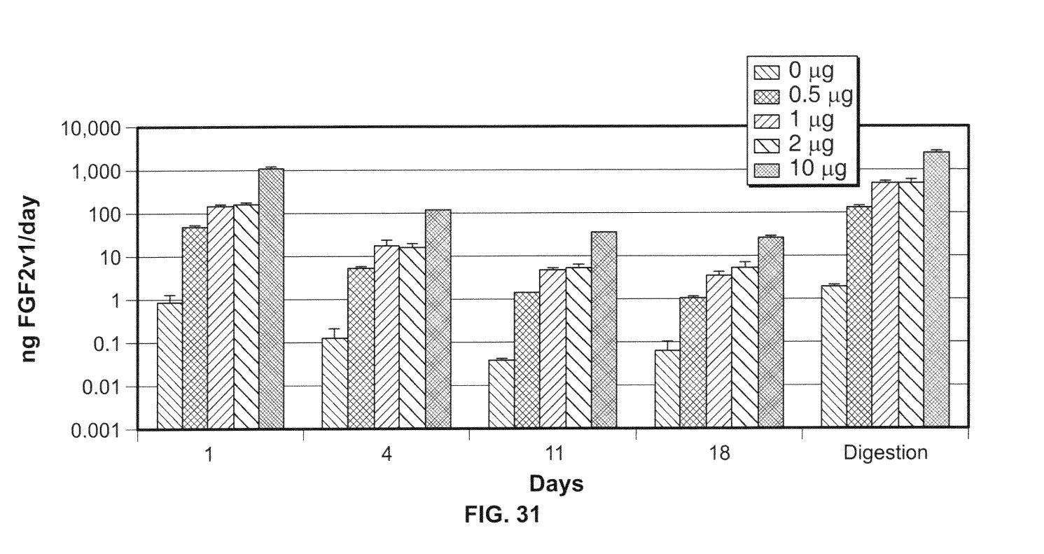

FIG. 31 presents the release of FGF2v1 from a combination of goat cartilage powder and a fibrinogen scaffold over time. Samples were tested at five initial dosages of FGF2v1 per ml of cartilage powder: 0.5 μg; 1 μg; 2 μg; 10 μg; and 0 μg (control). FGF2v1 was added to the cartilage powder in thrombin solution. Fibrin was added to form a fibrin matrix from which FGF2v1 release was measured. Data represent the daily released amounts of FGF2v1 from the four groups and the control group. Release of FGF2v1 was observed for at least 18 days. The amount of FGF2v1 released from the scaffold increased with the amount of FGF2v1 applied to the scaffold. A substance in the control sample reacted as FGF2v1 in the assay used.

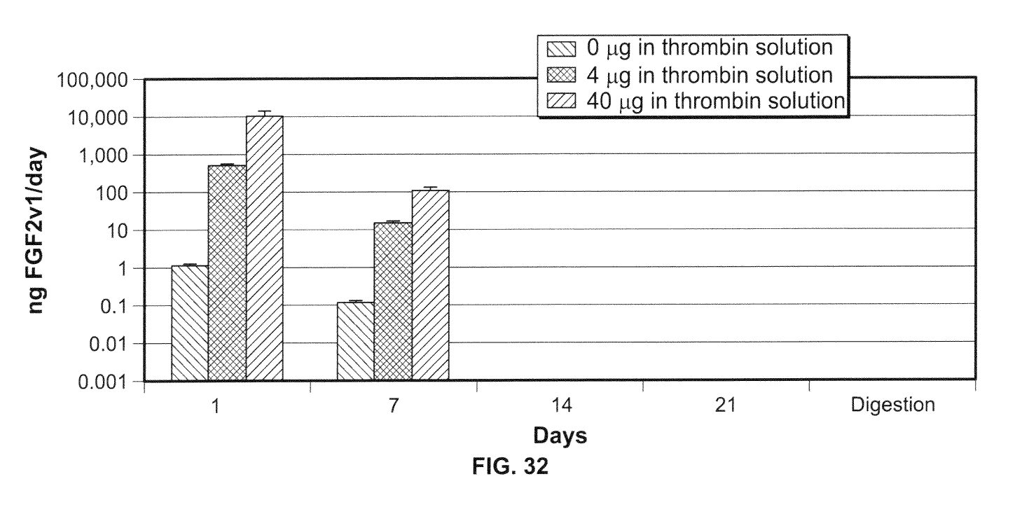

FIG. 32 presents the release of FGF2v1 from a combination of goat cartilage powder and a fibrinogen scaffold over time. Samples were tested at three initial dosages of FGF2v1 in thrombin solution: 4 μg/ml cartilage powder; 40 μg/ml; and 0 μg (control). Data represent the daily released amounts of FGF2v1 from the four groups and the control group. Release of FGF2v1 was observed for at least 7 days. The amount of FGF2v1 released from the scaffold increased with the amount of FGF2v1 applied to the scaffold. A substance in the control sample reacted as FGF2v1 in the assay used.

Based on the studies and data discussed above and illustrated in FIGS. 30-32, the following conclusions may be drawn regarding the combination of a scaffold, cartilage paste and FGF2v1:

(a) The scaffold physically stabilizes the cartilage paste to enable in-vivo transplantation.

(b) The cartilage paste/scaffold combination exhibits a slow release of FGF2v1 over an extended period of time.

(c) Variability was observed between the results of goat tissue studies and studies involving human tissue scaffolds.

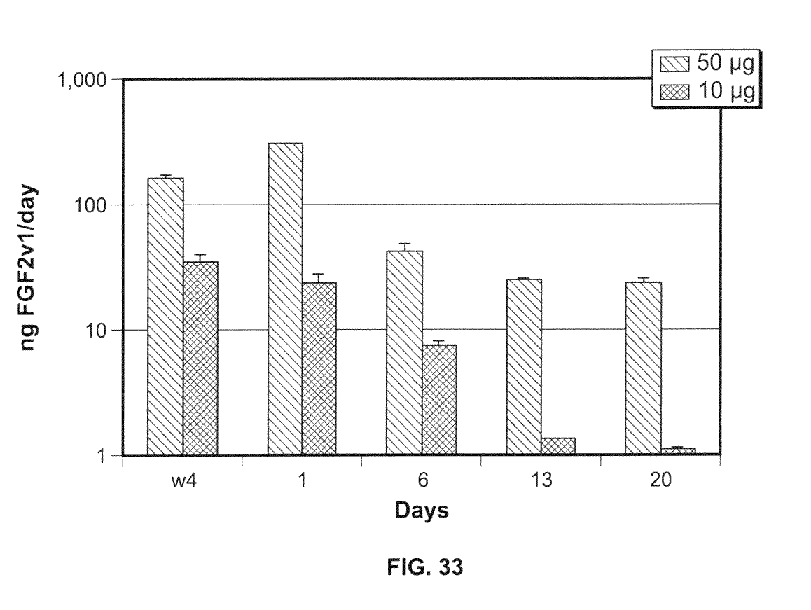

FIG. 33 presents the release of FGF2v1 from two groups of FGF2v1-loaded cancellous bone samples over time. The two groups included cancellous bone samples loaded with 50 μg of FGF2v1 and cancellous bone samples loaded with 10 μg of FGF2v1. Data represent the daily released amounts of FGF2v1 from the two groups. Release of FGF2v1 was observed for at least 20 days.

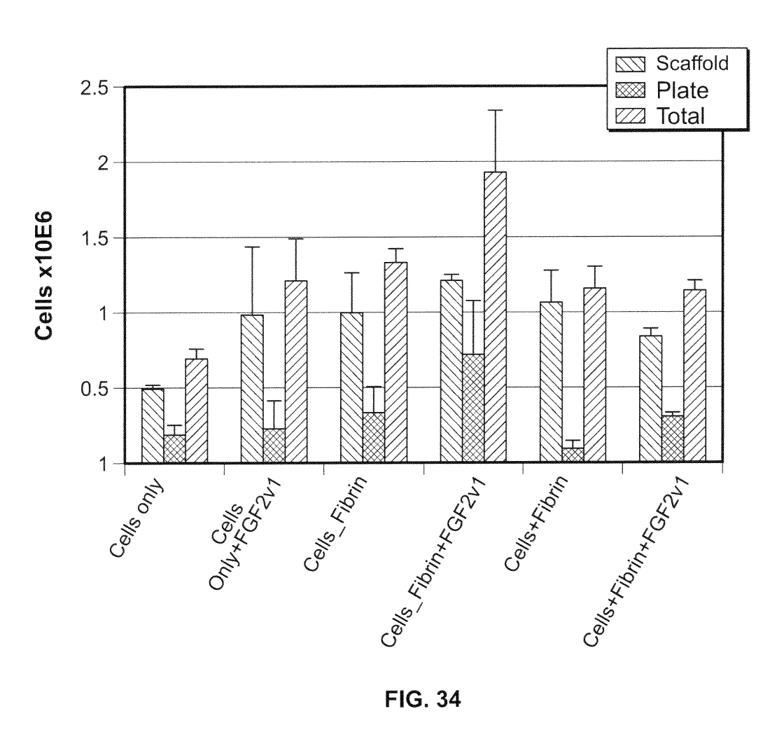

FIG. 34 presents the results of seeding human distal femur articular chondrocytes on cancellous bone strips. Six groups of cell solutions were seeded on both a scaffold and a plate. The six groups included cells only; cells with FGF2v1 added; cells in a fibrin matrix; cells in a fibrin matrix with FGF2v1 added; cells with fibrin added; and cells with fibrin and FGF2v1 added. Cell growth was measured on the scaffold and on the plate.

While a number of embodiments of the present invention have been described, it is understood that these embodiments are illustrative only, and not restrictive, and that many modifications and/or alternative embodiments may become apparent to those of ordinary skill in the art. For example, any steps may be performed in any desired order (and any desired steps may be added and/or any desired steps may be deleted). Therefore, it will be understood that the appended claims are intended to cover all such modifications and embodiments that come within the spirit and scope of the present invention.

We claim:

1. A sustained release composition, comprising lyophilized freeze-milled cartilage particles having a size no greater than 1 mm and an active growth factor exogenous to said cartilage particles, said growth factor being incorporated into said composition such that said growth factor is released substantially continuously over a prolonged period of time.

2. The composition of claim 1, further comprising a biocompatible carrier.

3. The composition of claim 2, wherein said biocompatible carrier is biodegradable.

4. The composition of claim 2, wherein said biocompatible carrier includes freeze-dried fibrin.

5. The composition of claim 4, wherein said fibrin is in the form of a matrix having open pores.

6. The composition of claim 4, wherein said fibrin is selected from the group consisting of human plasma protein, non-human mammalian plasma protein, avian plasma protein, recombinant plasma protein, partially-purified plasma protein, and totally purified plasma protein.

7. The composition of claim 1, wherein said cartilage particles have a size less than 210 microns.

8. The composition of claim 7, wherein said cartilage particles have a size in the range of from about 10 microns to about 210 microns, and constitute from about 10% to about 80% by weight of the composition.

9. The composition of claim 1, wherein said cartilage particles are selected from the group consisting of allograft cartilage, autograft cartilage and xenograft cartilage.

10. The composition of claim 9, wherein said cartilage particles are allograft cartilage.

11. The composition of claim 1, wherein said prolonged period of time is in the range of at least 2 days to about 10 days.

12. The composition of claim 11, wherein said growth factor is reversibly bound to said cartilage particles such that said growth factor is released substantially continuously into an aqueous medium in vitro over said prolonged period of time.

13. The composition of claim 1, wherein said prolonged period of time is in the range of at least 10 days to about 100 days.

14. The composition of claim 13, wherein said growth factor is reversibly bound to said cartilage particles such that said growth factor is released substantially continuously into an aqueous medium in vitro over said prolonged period of time.

15. The composition of claim 1, wherein said prolonged period of time is in the range of at least 2 days to about 120 days.

16. The composition of claim 15, wherein said growth factor is reversibly bound to said cartilage particles such that said growth factor is released substantially continuously into an aqueous medium in vitro over said prolonged period of time.

17. The composition of claim 1, wherein said prolonged period of time in the range of at least 2 days to about 180 days.

18. The composition of claim 17, wherein said growth factor is reversibly bound to said cartilage particles such that said growth factor is released substantially continuously into an aqueous medium in vitro over said prolonged period of time.

19. The composition of claim 1, wherein said growth factor is released into an aqueous medium in vitro such that the concentration of said growth factor in said aqueous medium varies by a factor of less than about 10 over said prolonged period of time.

20. The composition of claim 1, wherein said composition has an initial concentration of said exogenous growth factor in the range of about 0.5 μg per ml of said composition to about 5,000 μg per ml of said composition.

21. The composition of claim 1, wherein said growth factor is selected from the group consisting of native, recombinant and variant isoforms of FGF-2, FGF-4, FGF-5, FGF-7, FGF-9, FGF-11, FGF-21, TGF-β, BMP-2, BMP-4, BMP-7, BMP-14, PDGF, VEGF, and IGF-1.

22. The composition of claim 1, wherein said growth factor is selected from the group consisting of FGF isoforms having a least one substitution of an amino acid in the β8-β9 loop.

23. The composition of claim 22, wherein said at least one substitution is a single point substitution.

24. The composition of claim 1, wherein said growth factor is selected from the group consisting of native, recombinant and variant isoforms of FGF-2.

25. The composition of claim 1, wherein said growth factor is a human FGF-2.

26. The composition of claim 1, wherein said growth factor is a variant of a native human FGF-2, said variant having a substitution of amino acid 111 from asparagine to an amino acid selected from the group consisting of glycine, arginine, valine, glutamic acid and serine and is SEQ ID NO: 1.

27. The composition of claim 1, wherein said growth factor is a variant of a native human FGF-2, said variant having a substitution of amino acid 111 from asparagine to glycine and is SEQ ID NO: 3.

28. The composition of claim 1, wherein said growth factor is a variant of a native human FGF-2, said variant having a substitution of amino acid 3 from alanine to glutamine, a substitution of amino acid 5 from serine to glutamine, and a substitution of amino acid 111 from asparagine to an amino acid selected from the group consisting of glycine, arginine, valine, glutamic acid and serine and is SEQ ID NO: 4.

29. The composition of claim 1, wherein said growth factor is a variant of a native human FGF-2, said variant having a substitution of amino acid 3 from alanine to glutamine, a substitution of amino acid 5 from serine to glutamine, and a substitution of amino acid 111 from asparagine to glycine and is SEQ ID NO: 5.

30. The composition of claim 1, wherein said growth factor is selected from the group consisting of native, recombinant and variant isoforms of FGF-9.

31. The composition of claim 1, wherein said growth factor is a human FGF-9.

32. The composition of claim 1, wherein said growth factor is a variant of a native human FGF-9, said variant having a 63 amino acid N-terminus truncation and a substitution of amino acid 144 from tryptophan to an amino acid selected from the group consisting of glycine, arginine, glutamic acid and valine, and is SEQ ID NO: 6.

33. The composition of claim 1, wherein said growth factor is a variant of a native human FGF-9, said variant having a 63 amino acid N-terminus truncation, an 18 amino acid C-terminus truncation, and a substitution of amino acid 144 from tryptophan to an amino acid selected from the group consisting of glycine, arginine, glutamic acid and valine, and is SEQ ID NO: 7.

34. The composition of claim 1, wherein said growth factor is a variant of a native human FGF-9, said variant having a 63 amino acid N-terminus truncation and a substitution of amino acid 143 from asparagine to serine, and is SEQ ID NO: 8.

35. The composition of claim 1, wherein said growth factor is a variant of a native human FGF-9, said variant having a 63 amino acid N-terminus truncation, an 18 amino acid C-terminus truncation, and a substitution of amino acid 143 from asparagine to serine, and is SEQ ID NO: 9.

36. The composition of claim 4, wherein said fibrin is obtained by mixing plasma proteins comprising fibrinogen and Factor XIII with thrombin, and wherein said composition includes at least one anti-fibrinolytic agent.

37. The composition of claim 2, wherein said biocompatible carrier comprises a carrier selected from the group consisting of phosphate buffered saline solution, saline solution, sodium hyaluronate solution, hyaluronic acid and its derivatives, gelatin, collagen, chitosan, alginate, dextran, carboxymethylcellulose, and hydroxypropyl methylcellulose.

38. The composition of claim 37, wherein said biocompatible carrier is selected from the group consisting of sodium hyaluronate solution, hyaluronic acid and its derivatives and phosphate buffered saline solution.

39. A cartilage repair construct, comprising a demineralized bone portion which contains the composition of claim 1.

40. A method of repairing a defect in cartilage in a subject in need thereof, comprising the step of placing, in a defect site, a sustained release composition including lyophilized freeze-milled cartilage particles having a size no greater than 1 mm and an active growth factor exogenous to said cartilage particles, said growth factor being incorporated into said composition such that said growth factor is released substantially continuously over a prolonged period of time.

41. The method of claim 40, wherein the defect site is in articular cartilage.

42. The method of claim 40, wherein said step of placing the sustained release composition in a defect site at least partially relieves the need for a prosthetic replacement of a body part in which the defect site is located.

43. The method of claim 40, wherein said growth factor is reversibly bound to said cartilage particles such that said growth factor is released substantially continuously over a period of time.

44. A composition, comprising lyophilized freeze-milled cartilage particles having a size no greater than 1 mm and a biocompatible carrier including freeze-dried fibrin.
